(19) United States
(12) Patent Application Publication
(10) Pub. No.: US 2026/0115460 A1
(43) Pub. Date: Apr. 30, 2026

(54) IMPLANTABLE MEDICAL DEVICE

(71) Applicant: Medtronic, Inc., Minneapolis, MN (US)

(72) Inventors: Martin G. Hieb, St. Louis Park, MN (US); Kevin L. Sack, New Brighton, MN (US); Shangqian Peter Zhang, Shoreview, MN (US); Danielle Samone Rasmussen, North Branch, MN (US)

(21) Appl. No.: 19/366,650

(22) Filed: Oct. 23, 2025

Related U.S. Application Data
(60) Provisional application No. 63/711,938, filed on Oct. 25, 2024.

(51) Int. Cl. A61N 1/05; A61N 1/365; A61N 1/39

(52) U.S. Cl. CPC A61N 1/059; A61N 1/365; A61N 1/3956; A61N 1/3987

(57) ABSTRACT

A medical system including an implantable medical device (IMD) configured to position within a heart of a patient. The IMD includes a first helical body and a second helical body configured to travel into a tissue wall when the IMD is rotated in a first rotational direction. The second helical body may travel into the tissue as the first helical body travels into the tissue. In examples, at least one of the first helical body, the second helical body, or a housing of the IMD supports an electrode. The medical system may include processing circuitry configured to at least one of deliver therapy to the patient or sense a signal produced by the patient using the electrode.

### RELATED APPLICATIONS

This application claims the benefit of U.S. Provisional Application Serial Number 63/711,938, filed October 25, 2024, the entire contents of which are incorporated herein by reference.

### TECHNICAL FIELD

This disclosure is related to an implantable medical device.

### BACKGROUND

Implantable medical devices are often placed in a subcutaneous pocket and coupled to one or more transvenous medical electrical leads carrying pacing and sensing electrodes positioned in the heart. Intracardiac pacemakers have recently been introduced that are implantable within a ventricular chamber of a patient’s heart for delivering ventricular pacing pulses without the use of electrical leads. Such pacemakers or other implantable medical devices may also be able to detect the occurrence of arrhythmias, such as fibrillation, tachycardia and bradycardia, in the patient’s heart. An implantable cardiac defibrillator may deliver electrical shocks to the patient’s heart in response to detection of a tachycardia or fibrillation to restore a normal heartbeat in the patient. In some cases, a single implantable medical device functions as both an implantable pacemaker and implantable cardiac defibrillator.

Implantable medical devices may include electrodes and/or other elements for physiological sensing and/or therapy delivery. The electrodes and/or other elements may be implanted at target locations selected to detect a physiological condition of the patient and/or deliver one or more therapies. For example, the electrodes and/or other elements may be delivered to a target location within an atrium or ventricle to sense intrinsic cardiac signals and deliver pacing or antitachyarrhythmia shock therapy from a medical device coupled to a lead.

### SUMMARY

This disclosure describes a medical system including an implantable medical device (IMD) configured to position within a heart of a patient. The IMD may be configured to implant at least one electrode within tissue of a patient, e.g., cardiac tissue, such as a septal wall of the heart. The IMD is configured to position at least partially within a heart of a patient, such as within an atrium, ventricle, coronary sinus, or other portions of the heart. The IMD includes a first helical body and a second helical body configured to engage tissues to fixate the IMD within the patient. The first helical body and the second helical body surround an axis defined by the IMD, such that the second helical body may engage the tissues as the first helical body engages the tissues. The IMD may be configured such that the first helical body and the second helical body impart countering forces on the tissue as the first helical body and the second helical body implant within the tissue. The countering forces may ease an implantation of the IMD (e.g., by a clinician) and/or provide holding forces for the IMD once implanted.

In examples, a medical device configured to be positioned within an anatomical volume defined by a body of a patient comprises: a housing configured to be positioned within an anatomical volume defined by a body of a patient, the housing defining a housing distal portion and a defining a device axis extending through the housing distal portion, wherein the housing distal portion defines a housing distal end; a first helical body configured to engage tissues, the first helical body extending in a distal direction from the housing distal portion to a first distal end distal to the housing distal end, the first helical body surrounding the device axis, and the first helical body defining a helical handedness, the handedness being one of a right-handedness or a left handedness; a second helical body configured to engage tissues as the first helical body engages the tissues, the second helical body extending in the distal direction from the housing distal portion to a second distal end distal to the housing distal end, the second helical body surrounding the device axis, and the second helical body defining the helical handedness; and processing circuitry supported by the housing, wherein the processing circuitry is configured to at least one of provide therapy to the patient or sense a signal from the patient using an electrode supported by one of the first helical body, the second helical body, or the housing.

In examples, a medical device configured to be positioned within an anatomical volume defined by a body of a patient comprises: a housing configured to be positioned within an anatomical volume defined by a body of a patient, the housing defining a housing distal portion and a defining a device axis extending through the housing distal portion, wherein the housing distal portion defines a housing distal end; a first helical body configured to engage tissues, the first helical body extending in a distal direction from a first base portion coupled to the housing distal portion to a first distal end distal to the housing distal end, the first helical body surrounding the device axis, and the first helical body defining a helical handedness, the handedness being one of a right-handedness or a left handedness; a second helical body configured to engage the tissues as the first helical body engages the tissues, the second helical body extending in the distal direction from a second base portion coupled to the housing distal portion to a second distal end distal to the housing distal end, the second helical body surrounding the device axis, and the second helical body defining the helical handedness, wherein the first helical body defines a first helical axis angularly displaced from the device axis by a first angle, and wherein the second helical body defines a second helical axis angularly displaced from the device axis by a second angle. The first angle and/or the second angle may be non-zero angles.

In an example, a technique comprises: engaging, using a first helical body, tissues of a body of a patient, the first helical body extending in a distal direction from a housing defining a housing distal portion and a defining a device axis extending through the housing distal portion, wherein the housing distal portion defines a housing distal end, and the first helical body extending from a first base portion coupled to the housing distal portion to a first distal end distal to the housing distal end, wherein the first helical body surrounds the device axis and the first helical body defines a helical handedness, the handedness being one of a right-handedness or a left handedness; engaging, using a second helical body, the tissues of the body of the patient, the second helical body extending in the distal direction from the housing, the second helical body extending from a second base portion coupled to the housing distal portion to a second distal end distal to the housing distal end, wherein the second helical body surrounds the device axis and the second helical body defines the helical handedness; and communicating, using processing circuitry configured to at least one of provide therapy to the patient or sense a signal from the patient, with an electrode supported by one of the first helical body, the second helical body, or the housing.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical system including an implantable medical device.

FIG. 2 is a schematic plan view of the implantable medical device.

FIG. 3 is another schematic plan view of the implantable medical device.

FIG. 4 is a schematic end view of the implantable medical device of FIG. 2.

FIG. 5 is a schematic illustration of a first helical body and a second helical body of the implantable medical device configured to provide an offset from a device axis of the implantable medical device.

FIG. 6 is a schematic illustration of a first helical body and a second helical body configured to provide another offset from the device axis.

FIG. 7 is a schematic end view of the first helical body and/or the second helical body defining an example configuration between a distal end and a supported portion.

FIG. 8 is a schematic end view of the first helical body and/or the second helical body defining an another example configuration between a distal end and a supported portion.

FIG. 9 illustrates an example technique for using the implantable medical device.

### DETAILED DESCRIPTION

This disclosure describes an implantable medical device (IMD) configured to implant within an anatomical volume of a patient for the delivery of therapy to the patient and/or the sensing of signals from the patient. The IMD may be configured to position at least partially within a heart of a patient, such as within an atrium, ventricle, coronary sinus, or other portions of the heart. In examples, the IMD is configured to implant within and/or establish contact between at least one electrode and tissue of the patient, such as a septal wall of the heart or any wall of a heart chamber. The IMD includes an attachment device extending distal to a housing of the IMD (“IMD housing”) to assist in implantation and/or contact between electrodes of the IMD and the tissues. In examples, the attachment device is configured to substantially fixate the IMD to the tissue when the IMD housing is rotated (e.g., by a clinician) about an axis defined by the IMD (“IMD axis”). In examples, the anatomical volume is within and/or defined by any of a right atrium (RA), right ventricle (RV), left atrium (LA), and/or left ventricle (LV) of a heart of the patient.

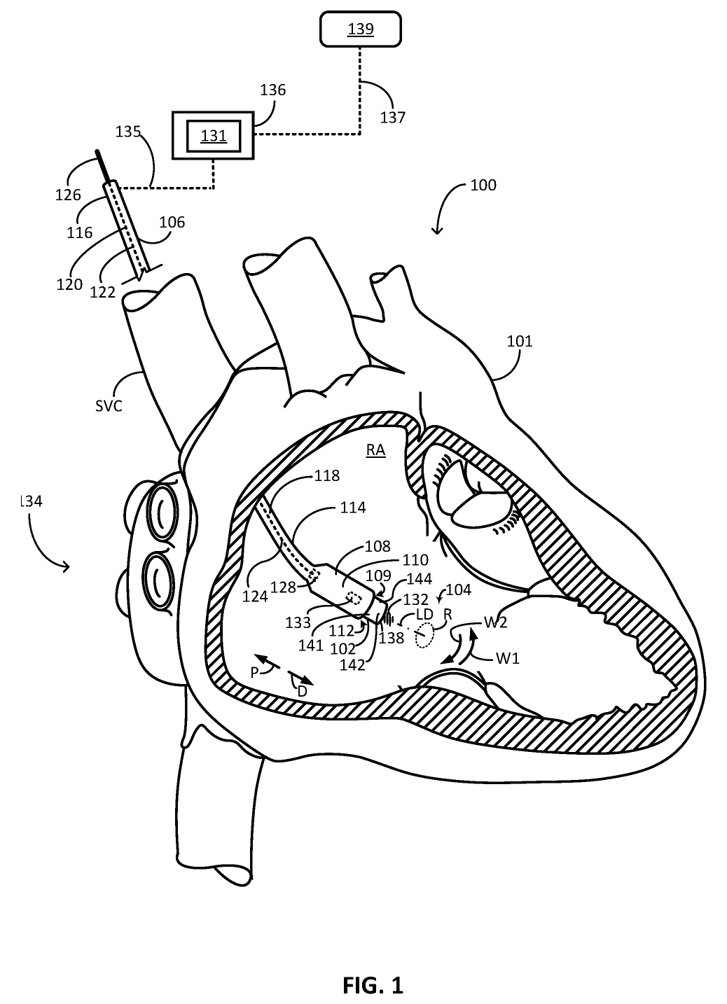

The IMD includes a first helical body and a second helical body, both extending distal to a distal portion of the IMD housing (“housing distal portion”). The first helical body extends to a first distal end configured to engage (e.g., penetrate) the tissue. The second helical body extends to a second distal end configured to engage (e.g., penetrate) the tissue. The first helical body and the second helical body may be configured to engage the tissue when the IMD housing is rotated about the IMD axis (e.g., the LD axis (FIGS. 1-6). In examples, the first helical body and the second helical body are configured to engage the tissue substantially concurrently when the IMD housing is rotated about the IMD axis. For example, the first helical body may be configured to implant into the tissue to a first depth as the IMD housing is rotated about the IMD axis. The second helical body may be configured to implant into the tissue to a second depth as the first helical body implants to the first depth (e.g., as the IMD housing is rotated about the IMD axis). The IMD may be configured such that the first helical body and the second helical body counter (e.g., substantially balance) forces transferred to the IMD housing during an implantation to, for example, assist in mitigating deviations from the intended orientation of the IMD during and/or following the implantation.

The first helical body defines a first helix surrounding a first helical axis. The second helical body defines a second helix surrounding a second helical axis. The second helical body and the second helical body define a similar and/or substantially the same helical handedness, such that both the first helical body and the second helical body travel in the tissue in the same direction when the IMD housing is rotated about the IMD axis. For example, in some examples, the first helical body and the second helical body each define a right-handed helix, such that rotation of the IMD housing in a clockwise direction (e.g., as viewed along the distal direction) causes the first helical body and the second helical body travel in the tissue in the distal direction. In some examples, the first helical body and the second helical body define a left-handed helix, such that rotation of the IMD housing in a counter-clockwise direction (e.g., as viewed along the distal direction) causes the first helical body and the second helical body travel in the tissue in the distal direction.

The IMD may be configured such that the first helical body and the second helical body substantially limit a deviation of the IMD axis from an initial orientation to a tissue surface as the IMD is implanted in the tissue. For example, the IMD may be initially oriented (e.g., prior to implantation) such that the IMD axis is generally pointed toward a particular region within a heart wall (e.g., a region including a His bundle, a portion of a bundle branch, other conduction system tissues, and/or or other tissues of the heart). The IMD may be configured such that, as the IMD housing is rotated about the IMD axis, the substantially concurrent travel of the first helical body and the second helical body into the tissue tend to limit deviation from the initial orientation, such that the IMD remains substantially oriented toward the particular region.

In examples, the IMD is configured to substantially balance forces received by the first helical body and the second helical body during implantation to limit the deviation. For example, the first helical body may be configured to receive a first force in a first direction from the tissue when the first helical body travels into the tissue. The first helical body may substantially transfer the first force to the IMD housing. The IMD may be configured to counter (e.g., substantially balance) this first force on the first helical body using the second helical body. For example, the IMD may be configured such that, as the second helical body travels substantially concurrently into the tissue, the second helical body receives a second force in a second direction from the tissue. The second helical body may substantially transfer the second force to the IMD housing. The second force in the second direction may tend to counter (e.g., to substantially balance) the first force in the first direction (and vice-versa), such that deviation from the initial orientation of the IMD is limited as the IMD fixates to the tissue wall. Hence, the first helical body and the second helical body may improve stability of the IMD as compared to a medical device having only a single helical body which, if acting alone, might transfer a single, un-countered force to a housing, potentially causing and/or exacerbating deviations of the medical device from an initial orientation. Furthermore, the stress carried by a single acting helical body induced by the IMD can be shared or lowered by the presence of dual acting helical bodies.

In examples, at least one of the first helical axis of the first helical body or the second helical axis of the second helical body is substantially offset (e.g., nonparallel) to the IMD axis, although this is not required. In some examples, both the first helical axis and the second helical axis are offset to the longitudinal axis. For example, the IMD may be configured such that the first helical axis and the second helical axis substantially lean inwards towards each other or substantially lean away from each other as the first helical body and the second helical body extend distal to the housing distal portion. In examples, the offsets of the first helical axis and the second helical axis assist in keeping an implanted IMD fixated to the tissue and/or mitigating departures from the initial orientation as the implanted IMD moves (e.g., during a heart beat) with the heart wall. In some examples, the offsets of the first helical axis and the second helical axis assist with balancing the forces received by the first helical body and the second helical body during IMD implantation.

In some examples, the IMD (e.g., the first helical body and/or the second helical body) may be configured such that the first helical axis and the second helical axis tend to reduce their respective offsets as the first helical body and the second helical body travel substantially concurrently within the tissues. For example, the first helical body and/or the second helical body may be configured such that the first helical axis and the second helical axis become closer to parallel with each other as the IMD fixates to the tissue wall. The first helical axis and the second helical axis becoming closer to parallel may cause the first helical body and/or the second helical body to impart forces against the tissue (e.g., forces substantially perpendicular to the first helical axis and/or the second helical axis) which tend to assist in keeping the implanted IMD fixated and/or mitigating departures from an initial orientation as an implanted IMD moves with the heart wall.

For example, a resilient biasing of the first helical body may cause the first helical body to impart a force on the tissue in a direction substantially perpendicular to the first helical axis as the first helical axis and the second helical axis become closer to parallel (e.g., as the IMD fixates to the tissue wall). The second helical body may substantially balance the force imparted by the first helical body by imparting a substantially equal and opposite force on the tissue. In examples, a resilient biasing of the second helical body causes the second helical body to impart the equal and opposite force. The force imparted by the first helical body and the force imparted by the second helical body may tend to assist in keeping the IMD fixated to the tissue and/or mitigate departures from the initial orientation. The first helical body and/or the second helical body may, in some examples, be configured such that the force imparted by the first helical body and the force imparted by the second helical body substantially point toward the IMD axis of the IMD (e.g., such that the first helical body and the second helical body substantially press inwards on the tissue). In some examples, the first helical body and/or the second helical body are configured such that the force imparted by the first helical body and the force imparted by the second helical body substantially point away from the IMD axis of the IMD (e.g., such that the first helical body and the second helical body substantially press outwards on the tissue).

In examples, one of the first helix, the second helix, and/or the IMD housing supports one or more electrodes configured to communicate with processing circuitry to at least one of deliver therapy to the patient or sense a signal produced by the patient. In some examples, the IMD housing may support the processing circuitry. In some examples, the IMD housing is configured to support the processing circuitry within an anatomical volume of the patient (e.g., a chamber of the heart) when the IMD housing positions within the anatomical volume. For example, the IMD housing may define a proximal portion (“housing proximal portion”) defining a proximal end (“housing proximal end”) opposite the housing distal end. The IMD may be configured such that the housing proximal end positions within the anatomical volume when the housing distal end positions within the anatomical volume. The IMD housing may support the processing circuitry substantially between the housing distal end and the housing proximal end, such that the IMD supports the processing circuitry within the anatomical volume when the housing positions within the anatomical volume. In other examples, the housing distal portion may be a portion of a medical lead configured to position within the anatomical volume and communicate with processing circuitry outside of the anatomical volume.

FIG. 1 is a conceptual diagram illustrating an example medical system 100 within a right atrium (“RA”) of a heart 101. Medical system 100 is configured to deliver and/or retrieve an implantable medical device 102 (“IMD 102”) to and/or from the vicinity of a target site 104 of heart 101. Medical system 100 may include a delivery catheter 106 supporting a receptacle device 108. Receptacle device 108 is configured to hold IMD 102 during delivery, deployment, and/or retrieval of IMD 102. Receptacle device 108 includes a receptacle wall 110 defining a receptacle volume 112 configured to hold and/or support IMD 102 during the delivery, deployment, and/or retrieval of IMD 102. In examples, receptacle device 108 defines an opening 109 (“receptacle opening 109”) which opens into receptacle volume 112. Receptacle opening 109 may be configured to allow at least IMD 102 to pass therethrough. In examples, medical system 100 is configured to deploy IMD 102 from receptacle device 108 (e.g., from a position within receptacle volume 112) and through receptacle opening 109 to cause IMD 102 to engage tissues within target site 104. In examples, medical system 100 is configured to cause IMD 102 to disengage from tissues within target site 104 to, for example, retrieve IMD 102 from and/or reposition IMD 102 within heart 101.

Delivery catheter 106 is configured to deliver receptacle device 108 and/or IMD 102 to an anatomical volume of the patient (e.g., the RA). Optionally, delivery catheter 106 may be advanced to the anatomical volume of the patient through a surrounding tubular member (not shown), such as a sheath or guide catheter, which may be placed with its distal end in the anatomical volume before delivery catheter 106 is advanced through the surrounding tubular member. In examples, delivery catheter 106 is configured to retrieve receptacle device 108 and/or IMD 102 from the anatomical volume of the patient. Delivery catheter 106 may include a distal portion 114 (“delivery catheter distal portion 114”) configured to be intracorporeal to the patient and a proximal portion 116 (“delivery catheter proximal portion 116”) which may be extracorporeal to the patient when delivery catheter distal portion 114 is intracorporeal. In examples, delivery catheter distal portion 114 supports (e.g., is attached to and/or is a substantially unitary component with) receptacle device 108. In examples, delivery catheter 106 is configured to deliver and/or retrieve receptacle device 108 and/or IMD 102 using vasculature of a patient, such as an SVC or other vasculature leading to the anatomical volume. In examples, delivery catheter 106 defines a lumen 118 (“delivery lumen 118”) which opens to receptacle volume 112.

In examples, medical system 100 includes a delivery system 120 configured to engage IMD 102 when, for example, IMD 102 is positioned within receptacle volume 112. Delivery system 120 may include a driver 122 that includes a distal portion 124 (“driver distal portion 124”) configured to be intracorporeal to the patient and a proximal portion 126 (“driver proximal portion 126”) which may be extracorporeal to the patient when driver distal portion 124 is intracorporeal. In examples, delivery system 120 includes a head portion 128 supported by driver distal portion 124 and configured to engage IMD 102. At least driver 122 may be configured to slidably translate and/or rotate within delivery lumen 118 (e.g., translate and/or rotate relative to delivery catheter 106). Delivery system 120 may be configured such that the translation and/or rotation of driver 122 causes driver 122 and/or head portion 128 to impart translational and/or rotational forces on IMD 102, such that IMD 102 translates and/or rotates relative to receptacle wall 110. For example, delivery system 120 may be configured to translate within delivery lumen 118 to impart (e.g., via head portion 128) a translational force on IMD 102 causing IMD 102 to translate (e.g., within receptacle volume 112) in a proximal direction P or a distal direction D relative to receptacle wall 110. Delivery system 120 may be configured to rotate within delivery lumen 118 to impart (e.g., via head portion 128) a rotational torque on IMD 102 causing IMD 102 to rotate (e.g., within receptacle volume 112) about an axis LD defined by IMD 102. In some examples, head portion 128 and driver 122 may be substantially separate components. In some examples, head portion 128 may be substantially contiguous with driver 122, such that head portion 128 and driver 122 define a unified component. In FIG. 1, portions of driver 122 and/or head portion 128 positioned within delivery lumen 118 and/or receptacle volume 112 are depicted with dashed lines.

A housing 141 of IMD 102 (“IMD housing 141”) includes a distal portion 142 (“housing distal portion 142”) defining a distal surface 138 (“IMD distal surface 138”). IMD distal surface may substantially surround and/or be intersected by an axis LD defined by IMD 102. In examples, IMD distal surface 138 includes one or more points defining a distal-most location of IMD housing 141. IMD 102 further includes an attachment device 132 configured to engage tissues within target site 104. Attachment device 132 is configured to extend distally (e.g., in distal direction D) from IMD distal surface 138 to assist in implantation of the IMD and/or assist in maintaining contact between electrodes of IMD 102 and tissues within target site 104. Attachment device 132 may be configured to secure IMD 102 to tissues of heart 101. In examples, IMD housing 141 mechanically supports attachment device 132. Attachment device 132 is configured to penetrate tissue of heart 101 at or near a target site, such as a target site 104. For example, attachment device 132 may be configured to penetrate cardiac tissue of or in an RV, RA, LV, and/or LA of heart 101, or penetrate cardiac tissue in another area of heart 101. Attachment device 132 may be configured to substantially maintain IMD 102 at or in the vicinity of the target site when attachment device 132 penetrates tissues at or in the vicinity of the target site.

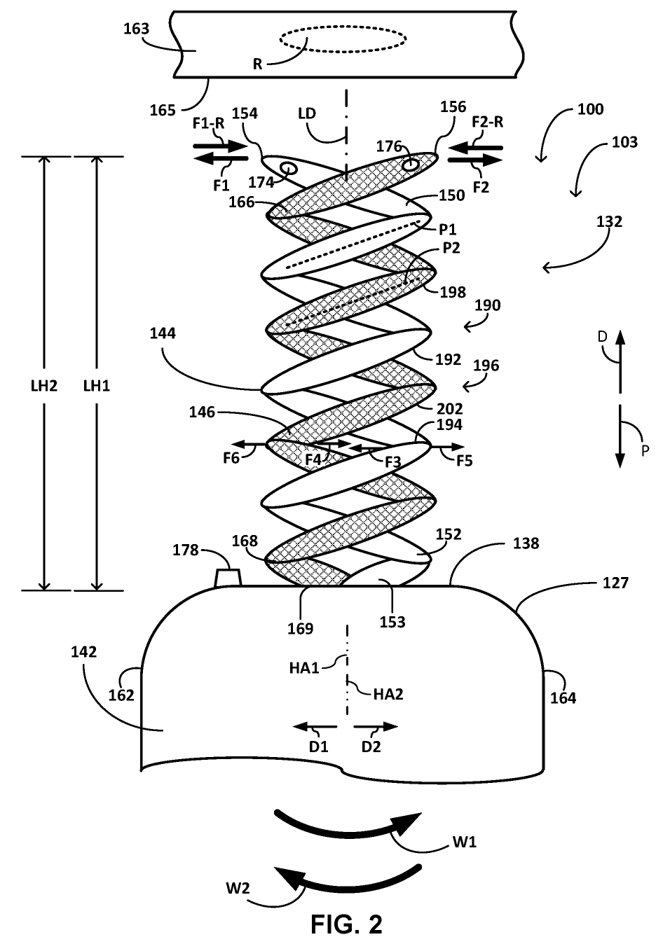

Attachment device 132 includes a first helical body (e.g., first helical body 144 (FIGS. 2-6) defining a distal end of the first helical body (e.g., first distal end 154 (FIGS. 2-6), and defines a second helical body (e.g., second helical body 146 (FIGS. 2-6) defining a distal end of the second helical body (e.g., second distal end 156 (FIGS. 2-6). In examples, the first helical body is configured such that the first distal end extends distal to housing distal portion 142 and/or IMD distal surface 138. The second helical body may be configured such that the second distal end extends distal to housing distal portion 142 and/or IMD distal surface 138. In examples, the first helical body and the second helical body are configured such that the first distal end and the second distal end are displaced distal to housing distal portion 142 and/or IMD distal surface 138 by substantially equal amounts.

The first helical body is configured to displace the first distal end within a tissue wall (e.g., at target site 104) when IMD housing 141 is rotated about axis LD in a particular rotational direction (e.g., one of a first rotary direction W1 or a second rotary direction W2). The second helical body is similarly configured to displace the secondary distal end within the tissue wall when IMD housing 141 is rotated in the particular rotational direction. For example, in some examples, the first helical body defines a first right-handed helix in a spatial coordinate system and the second helical body defines a second right-handed helix in the spatial coordinate system. In other examples, the first helical body defines a first left-handed helix in the spatial coordinate system and the second helical body defines a second left-handed helix in the spatial coordinate system. Hence, attachment device 132 is configured such that a rotation of IMD housing 141 in one of the first rotary direction W1 or second rotary direction W2 causes the first helical body and the second helical body to embed within the tissue wall, and a rotation of IMD housing 141 in the other of the first rotary direction W1 or second rotary direction W2 causes the first helical body and the second helical body to withdraw from the tissue wall.

IMD 102 may be configured such that the first helical body and the second helical body substantially counter respective forces transferred to IMD housing 141 during implantation to, for example, assist in mitigating deviations from an intended orientation of IMD 102 during and/or following the implantation. For example, in preparation for implantation at target site 104, IMD 102 may be initially oriented that axis LD is generally pointed toward a particular region R within a wall of heart 101. IMD 102 may be configured such that, as IMD housing 141 is rotated about axis LD and attachment device 132 implants within tissue, the substantially concurrent travel of the first helical body and the second helical body into the tissue tends to limit deviations from the initial orientation, such that the IMD remains substantially oriented toward region R.

In examples, attachment device 132 is configured such that the first helical body and the second helical body substantially balance forces that attachment device 132 transfers to IMD housing 141 during the implantation. For example, attachment device 132 may be configured such that the first helical body substantially transfers a first force to IMD housing 141 and the second helical body substantially transfers a second force countering (e.g., substantially balancing) the first force to IMD housing 141. The second force may counter the first force (and vice-versa), such that deviation from the initial orientation of IMD 102 is limited as IMD 102 fixates to the tissue wall. Hence, the first helical body and the second helical body may mitigate forces acting on IMD housing 141 during implantation which, if acting alone, might cause substantial deviation of axis LD during and/or following implantation from the initial orientation which was present prior to the implantation.

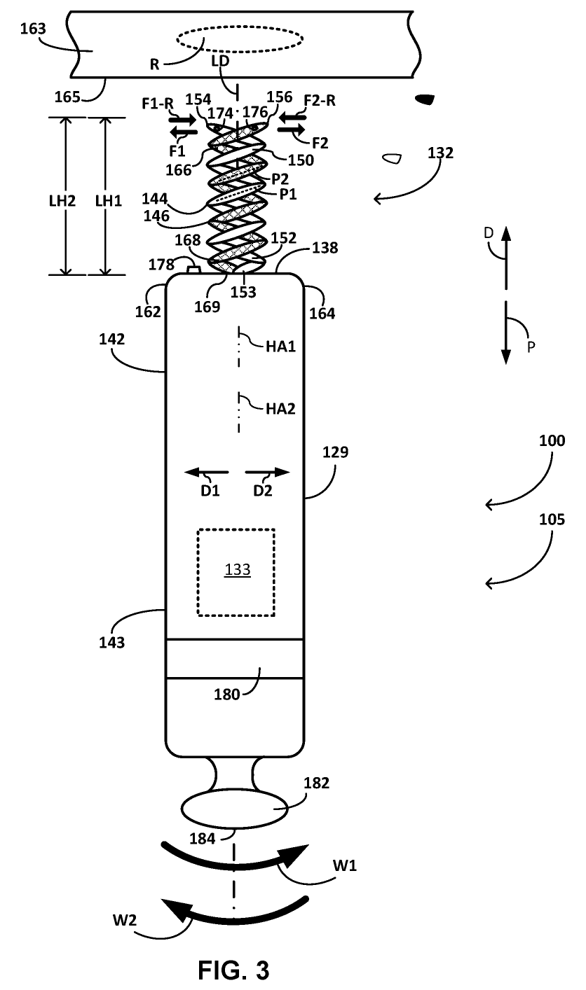

In examples, the first helical body supports a first electrode (e.g., first electrode 174 (FIGS. 2, 3, 5, 6) and/or the second helical body supports a second electrode (e.g., second electrode 176 (FIG. 2, 3, 5, 6). In some examples, instead of or in addition to the first electrode and/or the second electrode, IMD housing 141 (e.g., housing distal portion 142) supports a housing electrode (e.g., housing electrode 180 (FIG. 3). IMD 102 may include processing circuitry 133 configured to deliver therapy to and/or sense signals from a patient using the first electrode, the second electrode, and/or the housing electrode. In examples, the first helical body is configured to displace the first electrode within the tissue wall and/or the second helical body is configured to displace the second electrode within the tissue wall when IMD housing 141 is rotated in one of first rotary direction W1 or second rotary direction W2. The first helical body and second helical body may be configured such that a distance in which the first electrode and/or second electrode is embedded within the tissue wall is substantially proportional to an amount and direction of rotation of IMD housing 141. Hence, a clinician may control a depth a insertion of the first electrode and/or the second electrode based on a rotation of IMD housing 141. The clinician may control the depth of insertion using delivery system 120 (e.g., driver 122 and/or head portion 128) to impart a torque to IMD housing 141 in one of the first rotary direction W1 or second rotary direction W2.

In examples, delivery system 120 (e.g., head portion 128) is configured to engage IMD 102 to transfer a torque to IMD 102. Driver 122 may be configured to receive a torque (e.g., from a clinician) and transfer the torque to head portion 128. Delivery system 120 may be configured to engage with IMD 102 to, for example, implant IMD 102 within an anatomical volume, retrieve IMD 102 from an anatomical volume, re-position IMD 102 within an anatomical volume, and/or re-orient IMD 102 within an anatomical volume. In examples, IMD 102 includes one or more components (e.g., a communication antenna, a sensor, or another component) configured to rotate around and or revolve about axis LD when IMD 102 (e.g., IMD housing 141) rotates about axis LD. The first electrode, the second electrode, the housing electrode, and/or other electrodes of IMD 102 may be configured to establish electrical communication with tissues and/or other anatomical structures within heart 101 (e.g., tissues and/or other anatomical structures within target site 104).

Medical system 100 may be configured to position IMD 102 in proximity to target site 104 such that IMD 102 may be anchored to tissues within target site 104 (e.g., using attachment device 132). For example, delivery catheter 106 may be configured (e.g., under the influence of a clinician) to traverse vasculature of the patient to position receptacle device 108 and IMD 102 in proximity to target site 104. Medical system 100 (e.g., driver 122 and/or head portion 128) may be configured to impart a torque to IMD 102 to cause attachment device 132 to engage tissues (e.g., tissue within target site 104) when attachment device 132 is within or in proximity to target site 104. In examples, medical system 100 (e.g., driver 122 and/or head portion 128) is configured to impart a force (e.g., in the distal direction D) to IMD 102 to cause attachment device 132 to engage tissues (e.g., tissue within target site 104) when attachment device 132 is within or in proximity to target site 104. Medical system 100 may be configured such that driver 122 and/or head portion 128 may be disengaged from IMD 102 as IMD 102 remains anchored to tissues within or in proximity to target site 104. Delivery catheter 106, receptacle device 108, and delivery system 120 may subsequently be withdrawn from the patient (e.g., via vasculature of the patient).

In examples, medical system 100 is configured to deliver therapy to a patient and/or sense physiological signals of the patient received when receptacle device 108 is positioned (e.g., by a clinician) within heart 101. In some examples, medical system 100 may be configured to sense an indication of an intrinsic cardiac electrical signal produced by heart 101. Medical system 100 may be configured to process and/or condition a sensed signal to provide, for example, an indication of a location of receptacle device 108 and/or IMD 102 within heart 101, to conduct pace mapping for deployment of IMD 102, to provide indications indicative of the deployment and/or attachment of IMD 102 within heart 101, or for other reasons. In examples, medical system 100 includes processing circuitry 134 configured to deliver therapy and/or sense physiological signals. In examples, processing circuitry 134 includes processing circuitry such as device processing circuitry 131, configured to be mechanically supported by an external device 136 and/or another device of medical system 100. External device 136 and/or device processing circuitry 131 may be configured to be extracorporeal to the patient and/or otherwise displaced from receptacle device 108 and/or IMD 102 when, for example, receptacle device 108 and/or IMD 102 are intracorporeal to the patient. In examples, processing circuitry 134 includes processing circuitry such as IMD processing circuitry 133, configured to be mechanically supported by IMD 102 (e.g., IMD housing 141) and/or another device of medical system 100. IMD processing circuitry 133 may be configured to be intracorporeal to the patient when, for example, receptacle device 108 and/or IMD 102 are intracorporeal to the patient. Processing circuitry 134 may be configured to deliver therapy and/or sense physiological signals using one or more electrodes of medical system 100, such as the first electrode, the second electrode, another electrode supported by IMD 102, one or more electrodes supported by receptacle device 108, one or more electrodes supported by delivery system 120, an external electrode 139 configured to be extracorporeal to the patient, and/or one or more other electrodes in communication with and/or supported by medical system 100. In examples, processing circuitry 134 is configured to communicate with one or more electrodes using one or more communication links, such as communication link 135, communication link 137, and/or other communication links. In some examples, medical system 100 may include a snare configured to engage at least some portion of IMD 102 (e.g., IMD retrieval structure 182 (FIG. 3)).

Although the examples herein discuss delivery, retrieval, and/or positioning of IMD 102 within the RA of heart 101, medical system 100 may be configured to deliver, retrieve, and/or position IMD 102 in any of the other chambers of heart 101 and/or in other anatomical volumes of a patient in a like manner as that described for the RA of heart 101. Further, although the examples herein discuss attachment device 132 defining a first helical body and a second helical body, attachment device 132 may define other structures, such as one or more elongated tines extending from, for example, housing distal portion 142. Further, delivery system 120 may be configured to exert a translational force (e.g., in the distal direction D) on IMD 102 to, for example, cause and/or assist attachment device 132 in engaging tissues. Delivery system 120 may be configured to exert a translational force (e.g., in the proximal direction P) on IMD 102 to, for example, cause and/or assist attachment device 132 in disengaging from tissues. The translational force exerted by delivery system 120 may cause IMD 102 to move in the distal direction D or the proximal direction P relative to receptacle device 108, heart 101, and/or other portions of medical system 100. Target site 104 may include an appendage of the RA, a triangle of Koch region of the RA, a septal location in the RV or other location suitable to perform left bundle branch pacing (LBBP) or left bundle branch area pacing (LBBAP) via one or more electrodes (e.g. first electrode 174 and/or second electrode 176) supported by attachment device 132 at or near the distal end(s) of first and/or second helical bodies 144, 146, some other portion of heart 101, or some other location within a body of a patient.

Further, although some examples herein describe housing distal portion 142 comprising an IMD 102 wherein IMD housing 141 is configured to fit substantially entirely within an anatomical volume (e.g., a heart chamber) of a patient and/or be transported through vasculature of the patient, housing distal portion 142 is not so limited. In some examples, housing distal portion 142 may be a distal portion of medical lead supporting attachment device 132 (and/or one or more housing electrodes). The medical lead may be configured to communicate with processing circuitry 133 and/or other processing circuitry configured to reside outside the anatomical chamber (e.g., processing circuitry associated with a subcutaneously inserted pacemaker). For example, the medical lead may be configured to deliver therapy and/or sense physiological signals using one or more electrodes of medical system 100, such as the first electrode, the second electrode, the housing electrode, external electrode 139, and/or one or more other electrodes in communication with processing circuitry 133 and/or other processing circuitry of medical system 100.

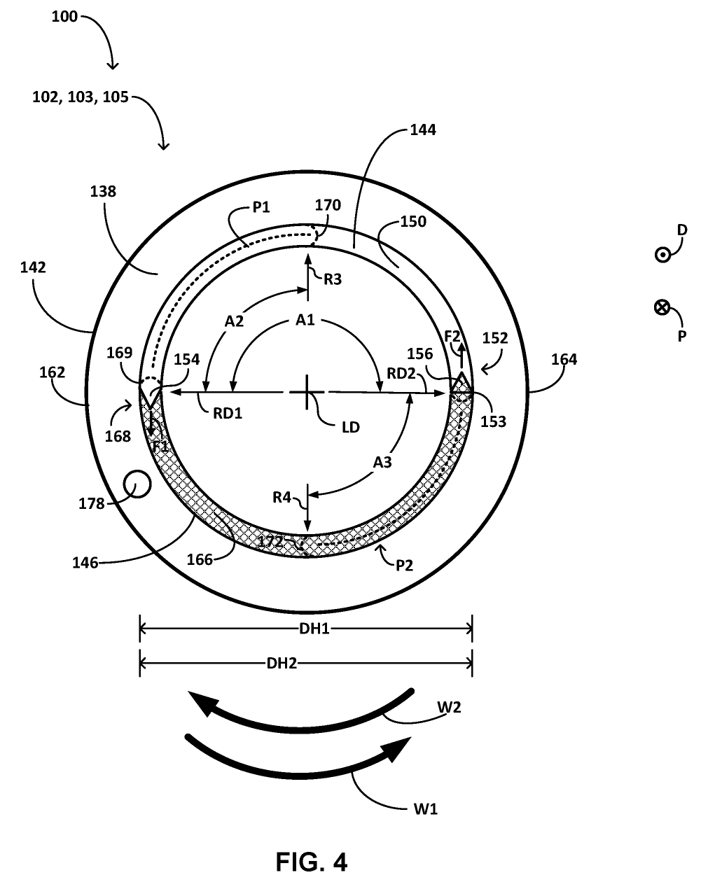

FIG. 2 is a schematic plan view of housing distal portion 142 of an IMD 103 including a first helical body 144 and a second helical body 146, with a distal direction D and a proximal direction P parallel to the page. IMD 103 includes IMD housing 127 defining housing distal portion 142. FIG. 3 is a schematic plan view of an IMD 105 including housing distal portion 142 and a proximal portion 143 (“housing proximal portion 143”) proximal to housing distal portion 142. IMD 103 includes IMD housing 129 defining housing distal portion 142. Housing proximal portion 143 defines a proximal end 149 (“housing proximal end 149”). In examples, IMD 105 is configured such that housing proximal end 149 positions within an anatomical volume (e.g., a heart chamber) when housing distal portion 142 positions within the anatomical volume. IMD 103, 105 is an example of IMD 102. IMD housing 127, 129 is an example of IMD housing 141. Axis LD extends through at least housing distal portion 142. In some examples (e.g., IMD 105), axis LD extends through housing distal portion 142 and housing proximal portion 143. In some examples, IMD 103 and/or IMD housing 127 is a portion of a medical lead. FIG. 4 is schematic end view of IMD 103, 105 including first helical body 144 and second helical body 146, with distal direction D proceeding out of the page and proximal direction P proceeding into the page.

In FIG. 2, FIG. 3, and FIG. 4, first helical body 144 is depicted as a helical body defining a helical path (e.g., helical path P1 (FIGS. 2-4) configured to advance distally when viewed along axis LD in distal direction D and rotated clockwise about axis LD (e.g., a first right-handed helix). Second helical body 146 is depicted as a helical body defining a helical path (e.g., helical path P2 (FIGS. 2-4, depicted as dashed white path) configured to advance distally when viewed along axis LD in distal direction D and rotated clockwise about axis LD (e.g., a second right-handed helix). However, it is understood that these specific orientations of first helical body 144 and second helical body 146 are not required and that the depictions and subsequent discussion are examples used consistently for clarity of illustration and discussion. For example, in some examples, first helical body 144 may be a first left-handed helix and second helical body 146 may be a second left-handed helix. In some examples, the “first rotational direction” discussed herein is one of first rotary direction W1 or second rotary direction W2 and the “second rotational direction” discussed herein is the other of first rotary direction W1 or second rotary direction W2.

IMD 102, 103, 105 is configured such that first helical body 144 and second helical body 146 tend to embed within a tissue wall 163 (e.g., via a tissue surface 165) when first helical body 144 and second helical body 146 are rotated in a first rotational direction (e.g., one of first rotary direction W1 or second rotary direction W2). IMD 102, 103, 105 is configured such that first helical body 144 and second helical body 146 tend to withdraw from tissue wall 163 when first helical body 144 and second helical body 146 are rotated in a second rotational direction (e.g., the other of first rotary direction W1 or second rotary direction W2). In examples, IMD 102. 103, 105 is configured such that first helical body 144 and second helical body 146 rotate about axis LD in a particular rotational direction when IMD housing 141, 127, 129 is rotated about and/or around about axis LD (e.g., by a clinician) in the particular rotational direction. For example, first helical body 144 and second helical body 146 may be configured to rotate about axis LD in first rotary direction W1 when IMD housing 141, 127, 129 rotates about axis LD in first rotary direction W1. First helical body 144 and second helical body 146 may be configured to rotate about axis LD in second rotary direction W2 when IMD housing 141, 127, 129 rotates about axis LD in second rotary direction W2. As used here, when first helical body 144 and/or second helical body 146 rotate about axis LD when IMD housing 141, 127, 129 rotates about axis LD, this may mean one or more fixed points defined by first helical body 144 and/or second helical body 146 undergo a substantially circular travel around axis LD as one or more fixed points defined by IMD housing 141, 127, 129 undergo a substantially circular travel around axis LD.

First helical body 144 extends at least from housing distal portion 142 (e.g., IMD distal surface 138) to a distal end 154 defined by first helical body 144 (“first distal end 154”). First distal end 154 may be configured to penetrate tissue wall 163 (e.g., tissue surface 165) when first distal end 154 is in contact with tissue wall 163 (e.g., tissue surface 165) and IMD housing 141, 127, 129 rotates in a particular direction about axis LD. Second helical body 146 extends at least from housing distal portion 142 (e.g., IMD distal surface 138) to a distal end 156 defined by second helical body 146 (“second distal end 156”). Second distal end 156 may be configured to penetrate tissue wall 163 (e.g., tissue surface 165) when second distal end 156 is in contact with tissue wall 163 (e.g., tissue surface 165) and IMD housing 141, 127, 129 rotates in the particular direction about axis LD. In examples, second helical body 146 is configured to implant within tissue wall 163 as first helical body 144 implants within tissue wall 163 (e.g., due to rotation of housing distal portion 142). In some examples, second distal end 156 is configured to penetrate and/or at least maintain contact with tissue wall 163 (e.g., tissue wall 163) when first distal end 154 penetrates tissue wall 163 (e.g., tissue surface 165). Similarly, first distal end 154 may be configured to penetrate and/or at least maintain contact with tissue wall 163 (e.g., tissue surface 165) when second distal end 156 penetrates tissue wall 163 (e.g., tissue surface 165).

First helical body 144 and second helical body 146 may be configured to transfer countering forces (e.g., substantially balanced forces) to IMD housing 141, 127, 129 (e.g., via housing distal portion 142) during an implantation in tissue wall 163. First helical body 144 and second helical body 146 may transfer the countering forces to, for example, assist in mitigating deviations from an intended orientation of housing distal portion 142 during and/or following an implantation. For example, in preparation for implantation within tissue wall 163, housing distal portion 142 may be initially oriented that axis LD is generally pointed toward particular region R within tissue wall 163. Housing distal portion 142 may be configured such that, as housing distal portion 142 rotates about axis LD and first helical body 144 and second helical body 146 (e.g., substantially concurrently) penetrate tissue surface 165 and/or implant within tissue wall 163, the countering forces transferred to housing distal portion 142 by first helical body 144 and second helical body 146 tend to limit deviations from the initial orientation (e.g., tend to keep housing distal portion 142 (e.g., IMD 102, 103, 105) remaining substantially oriented toward region R).

For example, housing distal portion 142 may be configured such that first helical body 144 imparts a first force F1 on tissue wall surface 165 as housing distal portion 142 rotates about axis LD and first distal end 154 penetrates tissue surface 165. First helical body 144 may be configured to receive a reaction force F1-R from tissue wall surface 165 in response to imparting first force F1. First helical body 144 may be configured to substantially transfer first reaction force F1-R to housing distal portion 142 (e.g., to IMD housing 141, 127, 129). Hence, force F1-R, if acting alone, might cause a movement of housing distal portion 142 (and/or first distal end 154) which causes axis LD to deviate from the initial orientation toward region R. In some cases, when force F1 and reaction force FR-1 result from the rotation of housing distal portion 142 about axis LD, force F1-R, when acting alone and in concert with the rotation about axis LD, may cause first distal end 154 to “wander” over tissue surface 165, potentially compromising the initial orientation of housing distal portion 142 relative to tissue wall 163.

Housing distal portion 142 (e.g., IMD 102, 103, 105) is configured to substantially counter first reaction force F1-R imparted to first helical body 144 using second helical body 146. Housing distal portion 142 may be configured such that second helical body 146 imparts a second force F2 on tissue wall surface 165 as first helical body 144 imparts first force F1. Second helical body 146 may be configured to receive a reaction force F2-R from tissue wall surface 165 in response to imparting second force F2. Second helical body 146 may be configured to substantially transfer second reaction force F2-R to housing distal portion 142 (e.g., to IMD housing 141, 127, 129). The transfer of second reaction force F2-R to housing distal portion 142 may tend to offset (e.g., to balance) the first reaction force F1-R transferred to housing distal portion 142, such that deviation of housing distal portion 142 from an initial orientation relative to tissue surface 165 may be mitigated.

In examples, first helical body 144 and second helical body 146 are configured such that at least some vector component of second force F2 has a direction opposite of first force F1. For example, referring largely to FIG. 4, first helical body 144 and second helical body 146 may be configured such that axis LD extends between first distal end 154 and second distal end 156. First helical body 144 and second helical body 146 may be configured such that at least some vector component of force F2 exerted by second distal end 156 is substantially equal and opposite in direction to force F1 exerted by first distal end 154. In some examples, the vector component comprises substantially the entirety of force F2, such that force F2 is substantially equal and opposite in direction to force F1.

In some examples, first helical body 144 and second helical body 146 are configured such that first distal end 154 positions in proximity to a first side 162 of housing distal portion 142 and second distal end positions in proximity to a second side 164 of housing distal portion 142 substantially opposite first side 162. For example, first helical body 144 and second helical body 146 may be configured such that first distal end 154 is displaced from axis LD by a first distal radius RD1 and second distal end 156 is displaced from axis LD by a second distal radius RD2. In examples, first distal radius RD1 and second distal radius RD2 subtend a distal arc angle A1 greater than about 120 degrees and less than about 240 degrees, in some examples, greater than about 145 degrees and less than about 215 degrees, and in some examples greater than about 170 degrees and less than about 190 degrees. In some examples, first distal radius RD1 and second distal radius RD2 subtend a distal arc angle A1 of about 180 degrees.

Hence, IMD 102, 103, 105 may be configured such that a clinician may position IMD 102, 103, 105 such that device axis LD has an initial orientation relative tissue surface 165 and/or region R. The clinician may impart a torque to IMD housing 141, 127, 129 in a particular rotational direction (e.g., one of first rotary direction W1 or second rotary direction W2) to cause first distal end 154 and second distal end 156 to penetrate tissue surface 165, such that force F1 imparted by first distal end 154 is countered (e.g., substantially balanced) by force F2 imparted by second distal end 156 during the rotation. The countering forces may limit and/or mitigate deviations from the initial orientation during the implantation.

First helical body 144 defines a distal portion 150 (“first distal portion 150”) and a base portion 152 (“first base portion 152”). First distal portion 150 defines first distal end 154. In examples, first base portion 152 extends from and/or is supported by housing distal portion 142. First helical body 144 is configured to extend in distal direction D beyond housing distal portion 142. For example, IMD 102, 103, 105 may be configured such that, when first base portion 152 extends from and/or is supported by housing distal portion 142, first distal portion 150 is displaced in distal direction D from IMD distal surface 138. IMD 102, 103, 105 may be configured such that, when first base portion 152 extends from and/or is supported by housing distal portion 142, first distal end 154 is displaced in distal direction D from IMD distal surface 138.

First helical body 144 may define first helical path P1 around axis LD. First helical path P1 may extend substantially from housing distal portion 142 (e.g. IMD distal surface 138) to first distal end 154. In examples, first helical path P1 defines a first helical length LH1 between housing distal portion 142 (e.g. IMD distal surface 138) and first distal end 154. In examples, first distal portion 150 extends from a midpoint of first helical path P1 to first distal end 154. First base portion 152 may substantially extend from housing distal portion 142 (e.g. IMD distal surface 138) to the midpoint of first helical path P1. In some examples, first base portion 152 extends from an intersection of first helical body 144 and a plane tangent to IMD distal surface 138 and substantially perpendicular to axis LD. In some examples, first base portion 152 extends from a supported portion 153 (“first supported portion 153”) supported by housing distal portion 142 and/or located at the intersection of first helical body 144 and the plane tangent to IMD distal surface 138.

First helical body 144 is configured such that first helical path P1 defines a first number of turns around a first helical axis such as first axis HA1. First helical body 144 may define first axis HA1. Although first axis HA1 is represented in FIG. 2 and FIG. 3 as a portion of first axis HA1 extending into housing 141, 127, 129 for clarity, first axis HA1 extends at least over first helical length LH1. In some examples, first helical body 144 is configured such that first axis HA1 is substantially coincident with or substantially parallel to device axis LD. In other examples, first helical body 144 may be configured such that first axis HA1 defines an angle with axis LD (e.g., either a positive angle (e.g., counter-clockwise relative to device axis LD) or a negative angle (e.g., clockwise relative to device axis LD). In examples, first helical body 144 defines a first helical diameter DH1 (FIG. 3). First helical diameter DH1 may be substantially perpendicular to first axis HA1. Second helical body 146 may define a second helical diameter DH2. In examples, second helical diameter DH2 is substantially equal to first helical diameter DH1, although this is not required.

First helical body 144 may define the first number of turns over the portion of first axis HA1 extending over first helical length LH1. In examples, first helical body 144 defines a first pitch of the first number of turns. In some examples, the first number of turns is indicative of a number of rotations of housing 141, 127, 129 which, following penetration of tissue surface 165 by first distal end 154, causes first helical body 144 to substantially fully implant into tissue wall 163. For example, the first number of turns may be indicative of a number of rotations of housing 141, 127, 129 which, following penetration of tissue surface 165 by first distal end 154, causes IMD distal surface 138 to substantially establish contact with tissue surface 165.

Second helical body 146 defines a distal portion 166 (“second distal portion 166”) and a base portion 168 (“second base portion 168”). Second distal portion 166 defines second distal end 156. In examples, second base portion 168 extends from and/or is supported by housing distal portion 142. Second helical body 146 is configured to extend in distal direction D beyond housing distal portion 142. For example, IMD 102, 103, 105 may be configured such that, when second base portion 168 extends from and/or is supported by housing distal portion 142, second distal portion 166 is displaced in distal direction D from IMD distal surface 138. IMD 102, 103, 105 may be configured such that, when second base portion 168 extends from and/or is supported by housing distal portion 142, second distal end 156 is displaced in distal direction D from IMD distal surface 138.

Second helical body 146 may define second helical path P2 around axis LD. Second helical path P2 may extend substantially from housing distal portion 142 (e.g. IMD distal surface 138) to second distal end 156. In examples, second helical path P2 defines a second helical length LH2 between housing distal portion 142 (e.g. IMD distal surface 138) and second distal end 156. In examples, second distal portion 166 extends from a midpoint of second helical path P2 to second distal end 156. Second base portion 156 may substantially extend from housing distal portion 142 (e.g. IMD distal surface 138) to the midpoint of second helical path P2. In some examples, second base portion 168 extends from an intersection of second helical body 146 and a plane tangent to IMD distal surface 138 and substantially perpendicular to axis LD. In some examples, second base portion 168 extends from a supported portion 169 (“second supported portion 169”) supported by housing distal portion 142 and/or located at the intersection of second helical body 146 and the plane tangent to IMD distal surface 138.

Second helical body 146 is configured such that second helical path P2 defines a second number of turns around a second helical axis such as second axis HA2. Second helical body 146 may define second axis HA2. Although second axis HA2 is represented in FIG. 2 and FIG. 3 as a portion of second axis HA2 extending into housing 141, 127, 129 for clarity, second axis HA2 extends at least over second helical length LH2. In some examples, second helical body 146 is configured such that second axis HA2 is substantially coincident with or substantially parallel to device axis LD and/or first axis HA1. In other examples, second helical body 146 may be configured such that second axis HA2 defines an angle with axis LD and/or first axis HA1 (e.g., either a positive angle (e.g., counter-clockwise relative to device axis LD and/or first axis HA1) or a negative angle (e.g., clockwise relative to device axis LD and/or first axis HA1). In examples, second helical body 146 may define a second helical diameter DH2 (FIG. 3). Second helical diameter DH2 may be substantially perpendicular to axis HA2. In examples, second helical diameter DH2 is substantially equal to first helical diameter DH1, although this is not required.

Second helical body 146 may define the second number of turns over the portion of second axis HA2 extending over second helical length LH2. In examples, second helical body 146 defines a second pitch of the second number of turns. In some examples, the second number of turns is indicative of a number of rotations of housing 141, 127, 129 which, following penetration of tissue surface 165 by second distal end 156, causes second helical body 146 to substantially fully implant into tissue wall 163. For example, the second number of turns may be indicative of a number of rotations of housing 141, 127, 129 which, following penetration of tissue surface 165 by second distal end 156, causes IMD distal surface 138 to substantially establish contact with tissue surface 165.

In examples, first helical body 144 and second helical body 146 are configured such that at least a first plurality of turns defined by first helical body 144 are substantially interleaved with a second plurality of turns defined by second helical body 146. For example, first helical body 144 may be configured such that each turn of the first plurality is separated from another turn of the first plurality by a turn of the second plurality. Second helical body 146 may be configured such that each turn of the second plurality is separated from another turn of the second plurality by a turn of the first plurality. For example, referring primarily to FIG. 3, first helical body 144 may define a first plurality of turns 190 (“first plurality 190”) including at least a turn 192 and a turn 194. Second helical body 146 may define a second plurality of turns 196 (“second plurality 196”) including at least a turn 198 and a turn 202. First plurality 190 may be substantially interleaved with second plurality 196 such that turn 202 of second plurality 196 separates turn 192 and turn 194 of first plurality 190, and/or such that turn 192 of first plurality 190 separates turn 198 and turn 202 of second plurality 196.

In examples, first helical body 144 and/or second helical body 146 are configured such that first helical length HA1 is substantially equal to second helical length HA2. First helical body 144 and/or second helical body 146 may be configured such that the first number of turns is substantially equal to the second number of turns, and/or such that the first pitch is substantially equal to the second pitch. In some examples, first helical body 144 is radially displaced from first axis HA1 over first helical length HA1 by a first radius substantially equal to first distal radius RD1 (e.g., first helical body 144 may define a first circular helix). Second helical body 146 may be radially displaced from second axis HA2 over second helical length HA2 by a second radius substantially equal to second distal radius RD2 (e.g., second helical body 146 may define a second circular helix).

In examples, first helical body 144 and/or second helical body 146 are configured to increase a travel of attachment device 132 into tissue wall 163 per rotation of IMD housing 141, 127, 129, as compared to an attachment member consisting of a single helical body. First helical body 144 and/or second helical body 146 may be configured to increase the travel while also implanting a substantially similar or increased number of turns within tissue wall 163 as the attachment member consisting of the single helical body. The increased number of turns implanted within tissue wall 163 may enhance the holding force of IMD 102, 103, 105 when attachment device 132 affixes to tissue wall 163. Hence, first helical body 144 and second helical body 146 may be configured to decrease an amount of rotations of housing 141, 127, 129 required to implant first helical body 144 and second helical body 146 as compared to the single helical body, while also providing a substantially similar or increased number of turns within tissue wall 163 as compared to the single helical body. Thus, first helical body 144 and second helical body 146 may reduce a number of turns required to be facilitated by a clinician to provide a satisfactory degree of holding force during an implantation procedure, as compared to the single helical body. Furthermore, in some examples, since helix to tissue friction is a surface area effect, first helical body 144 and second helical body 146 may provide more frictional force to hold 144 and 146 is realized in comparison to a single helical body.

In some examples, first helical body 144 and second helical body 146 may be configured to mitigate a bending (e.g., a lateral bowing) of first helical body 144 and/or second helical body 146 during and/or following an implantation of IMD 102, 103, 105. First helical body 144 and second helical body 146 may be configured to mitigate the bending as IMD housing 141, 127, 129 moves relative to tissue wall 163 and/or tissue surface 165 in a first direction D1 and/or a second direction D2. For example, first helical body 144 and second helical body 146 may be configured to mitigate the bending when a torque around a torque axis is imparted to IMD 102, 103, 105 (e.g., to cause rotation of IMD housing 141, 127, 129 during an implantation procedure), and deviations of the torque axis orientation relative to, for example, device axis LD, cause IMD housing 141, 127, 129 to move in the first direction D1 and/or the second direction D2. First helical body 144 and second helical body 146 may be configured to mitigate the bending when movement of tissue wall 163 (e.g., during a heartbeat) imparts force to IMD housing 141, 127, 129 causing the movement in the first direction D1 and/or the second direction D2. The mitigated bending may than act to lessen an amount of the movement of IMD housing 141, 127, 129 in the first direction D1 and/or the second direction D2.

For example, a helical body, such as the single helical body previously discussed, may define a number of turns about a helical axis defined by the helical body. The helical body may have a flexural rigidity which is indicative of a bending (e.g., a bowing) of the helical body relative to its helical axis when a force is imparted on the helical body (e.g., in a direction substantially perpendicular to the helical axis). The helical body may be configured such that the flexural rigidity decreases (e.g., the expected bowing increases) as the number of turns about the helical axis increases. Hence, increasing a number of turns defined by the helical body may increase an amount of bending which occurs when a given force is imparted to the helical body. Similarly, decreasing a number of turns defined by the helical body may decrease the amount of bending which occurs when the given force is imparted to the helical body. Thus, the single helical body previously discussed would be expected to experience bending depending on the number of turns about the helical axis defined by the single helical body (e.g., as the single helical body extends over first helical length HA1 or second helical length HA2).

First helical body 144 and/or second helical body 146 may be configured to mitigate the bending, as compared to the single helical body. As discussed above, first helical body 144 and second helical body 146 may be configured to, in combination, provide a substantially similar number of turns over first helical length HA1 and/or second helical length HA2 as compared to the single helical body. However, first helical body 144 and second helical body 146 may provide this substantially similar number of turns by substantially distributing the turns over first helical body 144 and second helical body 146. Hence, the first number of turns defined by first helical body 144 and/or the second number of turns defined by second helical body 146 may be less than the number of turns defined by the single helical body as first helical body 144 and second helical body 146, in combination, provide the substantially similar number of turns as the single helical body. Thus (e.g., due to the decreased number of turns), first helical body 144 and/or second helical body 146 may a flexural rigidity greater than the single helical body as first helical body 144 and/or second helical body 146 extend over first helical length HA1 and/or second helical length HA2. The increased flexural rigidity may mitigate the movement of IMD housing 141, 127, 129 in the first direction D1 and/or the second direction D2 (e.g., during and/or following an implantation of IMD 102, 103, 105).

In examples, at least one of first axis HA1 of first helical body 144 or second axis HA2 of second helical body 146 is substantially offset to axis LD (e.g., nonparallel or defining either a positive angle (e.g., counter-clockwise relative to device axis LD or a negative angle (e.g., clockwise relative to device axis LD). In some examples, both first axis HA1 and second axis HA2 are offset to axis LD. In some examples, the offsets of first axis HA1 and second axis HA2 may assist in keeping housing distal portion 142 fixated to tissue wall 163 and/or mitigating departures from an initial orientation as housing distal portion 142 moves (e.g., during a heartbeat) with tissue wall 163. In some examples, first helical body 144 and/or second helical body 146 may tend to reduce their respective offsets as first helical body 144 and second helical body 146 travel substantially concurrently within tissues wall 163 (e.g., such that first axis HA1 and second axis HA2 become closer to parallel as first helical body 144 and second helical body 146 travel within tissues wall 163). The reduction of the offsets from axis LD and/or each other may cause first helical body 144 and/or second helical body 146 to impart forces against tissues of tissue wall 163 to assist in, for example, keeping IMD 102, 103, 105 fixated to tissue wall 163, and/or mitigating departures from an initial orientation as IMD 102, 103, 105 moves with tissue wall 163.

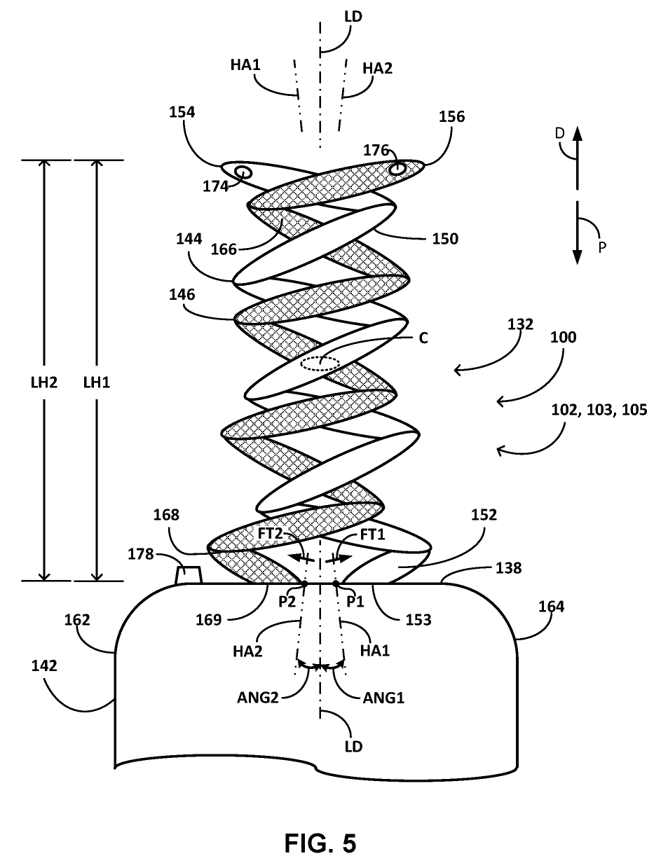

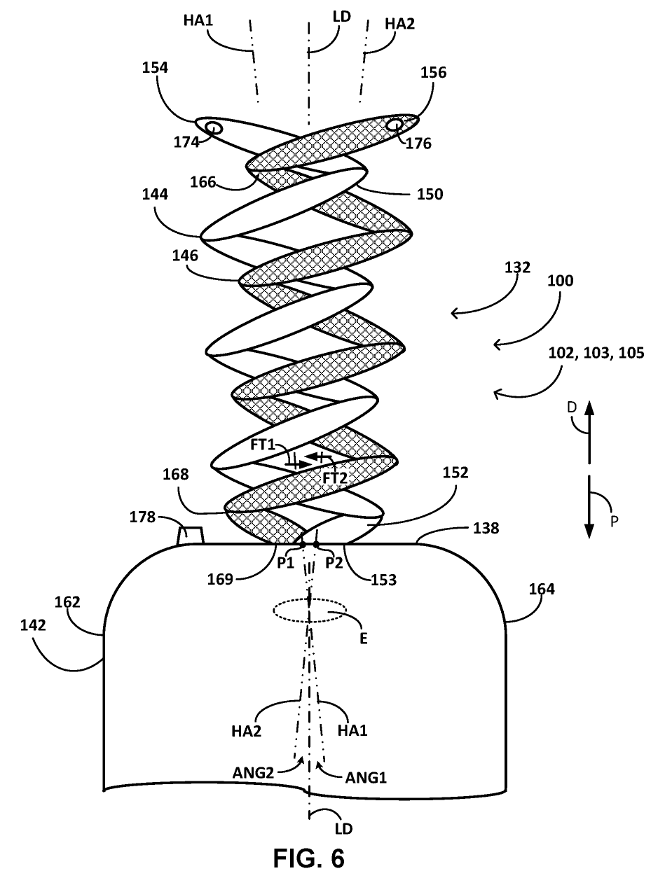

For example, FIG. 5 schematically illustrates housing distal portion 142 with first helical body 144 defining first axis HA1 and second helical body 146 defining second axis HA2. As depicted in FIG. 5, first helical body 144 and second helical body 146 may be configured (e.g., supported by housing distal portion 142) such that first axis HA1 and/or second axis HA2 substantially lean inwards towards axis LD and/or each other as first helical body 144 and second helical body 146 initially extend distal to housing distal portion 142. FIG. 6 schematically illustrates housing distal portion 142 with first helical body 144 and second helical body 146 configured (e.g., supported by housing distal portion 142) such that first axis HA1 and/or second axis HA2 substantially lean outwards away from axis LD and/or each other as first helical body 144 and second helical body 146 extend distal to housing distal portion.

For example, referring largely to FIG. 5, first helical body 144 may be configured such that first axis HA1 and axis LD define an angle ANG1 as first base portion 152 extends from housing distal portion 142 (e.g., IMD distal surface 138). First helical body 144 may be configured such that the angle ANG1 causes first base portion 152 to substantially lean towards axis LD and/or second base portion 168 as first base portion 152 extends from IMD distal surface 138 in distal direction D. In some examples, ANG1 is greater than or equal to about -30 degrees and less than or equal to about 30 degrees (e.g., relative to device axis LD), in some examples greater than or equal to about -15 degrees and less than or equal to about 15 degrees (e.g., relative to device axis LD). In some examples, ANG1 is greater than or equal to -10 degrees and less than or equal to about 10 degrees (e.g., relative to device axis LD).

In some examples, first helical axis HA and axis LD are substantially coplanar lines (e.g., first axis HA1 may intersect axis LD) and angle ANG1 is defined within a plane which includes first axis HA1 and axis LD, although this is not required. In some examples, first axis HA1 and axis LD may be substantially skew lines defining ANG1. In some examples, for example when first axis HA1 and axis LD are substantially skew lines, ANG1 is a maximum angle defined by first axis HA1 and axis LD.

First helical body 144 may be configured such that ANG1 causes first axis HA1 and axis LD to define a vertex of ANG1 distal to first supported portion 153 and/or IMD distal surface 138. For example, first helical body 144 and IMD 102, 103, 105 may be configured such that first axis HA1 and axis LD define the vertex of ANG1 within a region C distal to first supported portion 153 and/or IMD distal surface 138. In examples, region C lies substantially between first supported portion 153 and first distal end 154.

Second helical body 146 may be configured such that second axis HA2 and axis LD define an angle ANG2 as second base portion 168 extends from housing distal portion 142 (e.g., IMD distal surface 138). Second helical body 146 may be configured such that the angle ANG2 causes second base portion 168 to substantially lean towards axis LD and/or first base portion 152 as second base portion 168 extends from IMD distal surface 138 in distal direction D. In some examples, ANG2 is greater than or equal to -30 degrees and less than or equal to about 30 degrees (e.g., relative to device axis LD), in some examples greater than or equal to about -15 degrees and less than or equal to about 15 degrees (e.g., relative to device axis LD). In some examples, ANG2 is greater than or equal to about -10 degrees and less than or equal to about 10 degrees (relative to device axis LD).

In examples, second helical axis HA and axis LD are substantially coplanar lines (e.g., second axis HA2 may intersect axis LD) and angle ANG2 is defined within a plane which includes second axis HA2 and axis LD, although this is not required. In some examples, second axis HA2 and axis LD may be substantially skew lines defining ANG2. In some examples, for example when second axis HA2 and axis LD are substantially skew lines, ANG2 is a maximum angle defined by second axis HA2 and axis LD. In some examples, second axis HA2 and first axis HA1 are substantially coplanar lines (e.g., second axis HA2 may intersect first axis HA1), although this is not required. Second axis HA2 and first axis HA1 may be substantially skew lines

Second helical body 146 may be configured such that ANG2 causes second axis HA2 and axis LD to define a vertex of ANG2 distal to second supported portion 169 and/or IMD distal surface 138. For example, second helical body 146 and IMD 102, 103, 105 may be configured such that second axis HA2 and axis LD define the vertex of ANG2 within region C. In examples, region C lies substantially between second supported portion 169 and second distal end 156.

First helical body 144 and/or second helical body 146 may be configured such that first axis HA1 and/or second axis HA2 tend to reduce their respective offsets from axis LD as first helical body 144 and/or second helical body 146 travel (e.g., travel substantially concurrently) within tissue wall 163 (e.g., due to rotation of IMD housing 141, 127, 129). In examples, first helical body 144 and/or second helical body 146 are configured to exert forces on tissues within tissue wall 163 in a direction toward axis LD as first axis HA1 and/or second axis HA2 reduce their respective offsets from axis LD. The forces exerted by first helical body 144 and second helical body 146 may substantially act as holding forces tending to, for example, mitigate backing out of IMD 102, 103, 105 from tissue wall 163 once implanted, and/or tending to mitigate deviation of axis LD relative to region R (FIG. 2, FIG. 3).

For example, first helical body 144 and/or housing distal portion 142 may be configured such that first axis HA1 becomes closer to parallel with axis LD as first helical body 144 travels within tissue wall 163 (e.g., due to substantially concurrent travel of second helical body 146 into tissue wall 163). In examples, first helical body 144 and/or housing distal portion 142 are configured such that ANG1 decreases as first helical body 144 travels within tissue wall 163. First axis HA1 becoming closer to parallel with axis LD (e.g., ANG1 decreasing) may cause first helical body 144 to impart a force F3 (FIG. 2) against tissues of tissue wall 163. In examples, force F3 is substantially perpendicular to first axis HA1 and/or acts in a direction substantially toward device axis LD. In examples, force F3 acts in a direction substantially from first helical body 144 toward second helical body 146. Force F3 may be a vector component of a resultant force exerted by first helical body 144. In examples, first distal portion 150 is configured to displace away from axis LD as first axis HA1 becomes closer to parallel with axis LD and first helical body 144 commences exerting force F3.

Stated similarly, first helical body 144 and/or housing distal portion 142 may be configured such that first axis HA1 defines a first point P1 where first axis HA1 intersects IMD distal surface 138. First helical body 144 and/or housing distal portion 142 may be configured such that first axis HA1 rotates substantially about first point P1 (e.g., pivots about first point P1) when first helical body 144 causes helical axis HA1 to become closer to parallel with axis LD (e.g., as ANG1 decreases). The rotation substantially about first point P1 may cause first axis HA1 to rotate relative to IMD distal surface 138 in first traverse direction FT1 away from axis LD.

In the example of FIG. 5, with region C distal to first supported portion 153 and/or IMD distal surface 138, the vertex of ANG1 is such that first point P1 is displaced from axis LD in a direction toward second side 164 (e.g., displaced in the direction RD2 (FIG. 4)), and such that the movement of first axis HA1 in first traverse direction FT1 causes first axis HA1 to move away from axis LD. The movement of first axis HA1 away from axis LD may tend to cause first helical body 144 to exert force F3 in the direction substantially toward device axis LD.

In examples, first helical body 144 is resiliently biased to cause first axis HA1 to substantially maintain ANG1 in the absence of other forces acting on first helical body 144 (e.g., to resist the rotation in first traverse direction FT1). In examples, the resilient biasing tends to cause first helical body 144 to attempt to return to a configuration substantially maintaining ANG1 when external forces act on first helical body 144 to cause a departure from (e.g., a decrease of) ANG1. In examples, the resilient biasing of first helical body 144 cause a generation of internal forces within first helical body 144 which cause first helical body 144 to attempt to return to the configuration substantially maintaining ANG1. First helical body 144 may impart force F3 on tissues of tissue wall 163 as first helical body 144 experiences the internal forces caused by the resilient biasing (e.g., as the internal forces act against the rotation in first traverse direction FT1). Hence, the resilient biasing of first helical body 144 may cause first helical body 144 to impart force F3 as first axis HA1 becomes closer to parallel with axis LD, and/or as first helical body 144 bends outwards away from axis LD.

Second helical body 146 and/or housing distal portion 142 may be configured such that second axis HA2 becomes closer to parallel with axis LD as second helical body 146 travels within tissue wall 163 (e.g., due to substantially concurrent travel of first helical body 144 into tissue wall 163). In examples, second helical body 146 and/or housing distal portion 142 are configured such that ANG2 decreases as second helical body 146 travels within tissue wall 163. Second axis HA2 becoming closer to parallel with axis LD (e.g., ANG2 decreasing) may cause second helical body 146 to impart a force F4 (FIG. 2) against tissues of tissue wall 163. In examples, force F4 is substantially perpendicular to second axis HA2 and/or acts in a direction substantially toward device axis LD. In examples, force F4 acts in a direction substantially from second helical body 146 toward first helical body 144. Force F4 may be a vector component of a resultant force exerted by first helical body 144. In examples, second distal portion 166 is configured to displace away from axis LD as second axis HA2 becomes closer to parallel with axis LD and second helical body 146 commences exerting force F4.

Stated similarly, second helical body 146 and/or housing distal portion 142 may be configured such that second axis HA2 defines a second point P2 where second axis HA2 intersects IMD distal surface 138. Second helical body 146 and/or housing distal portion 142 may be configured such that second axis HA2 rotates substantially about second point P2 (e.g., pivots about second point P2) when second helical body 146 causes second axis HA2 to become closer to parallel with axis LD (e.g., as ANG2 decreases). The rotation substantially about second point P2 may cause second axis HA2 to rotate relative to IMD distal surface 138 in a second traverse direction FT2 away from axis LD.

In the example of FIG. 5, with region C distal to first supported portion 153 and/or IMD distal surface 138, the vertex of ANG2 is such that second point P2 is displaced from axis LD in a direction toward first side 162 (e.g., displaced in the direction RD1 (FIG. 4)), and such that the movement of second helical axis HA1 in second traverse direction FT2 causes second axis HA2 to move away from axis LD. The movement of first axis HA1 away from axis LD may tend to cause first helical body 144 to exert force F4 in the direction substantially toward device axis LD. In examples, first helical body 144 and/or second helical body 146 are configured such that second traverse direction FT2 is substantially opposite first traverse direction FT1. In some examples, first helical body 144 and/or second helical body 146 are configured such that axis LD is between first point P1 and second point P2.

In examples, second helical body 146 is resiliently biased to cause second axis HA2 to substantially maintain ANG2 in the absence of other forces acting on second helical body 146 (e.g., to resist the rotation in second traverse direction FT2). In examples, the resilient biasing of second helical body 146 tends to cause second helical body 146 to attempt to return to a configuration substantially maintaining ANG2 when external forces act on second helical body 146 to cause a departure from (e.g., a decrease of) ANG2. In examples, the resilient biasing of second helical body 146 cause a generation of internal forces within second helical body 146 which cause second helical body 146 to attempt to return to the configuration substantially maintaining ANG2. Second helical body 146 may impart force F4 on tissues of tissue wall 163 as second helical body 146 experiences the internal forces caused by the resilient biasing of second helical body 146 (e.g., as the internal forces act against the rotation in second traverse direction FT2). Hence, the resilient biasing of second helical body 146 may cause second helical body 146 to impart force F4 as second axis HA2 becomes closer to parallel with axis LD, and/or as second helical body 146 bends outwards away from axis LD.

In some examples, first helical body 144 and/or second helical body 146 may be configured to exert forces on tissues within tissue wall 163 in a direction away from device LD. In examples, first helical body 144 is configured to imparts a force F5 (FIG. 2) in a first direction away from device LD against tissues of tissue wall 163. In examples, second helical body 146 is configured to imparts a force F6 (FIG. 2) in a second direction away from device LD against tissues of tissue wall 163. In some examples, first helical body 144 is configured to exert force F5 and second helical body 146 exerts force F6.

For example, FIG. 6 schematically illustrates housing distal portion 142 with first helical body 144 and second helical body 146 configured (e.g., supported by housing distal portion 142) such that first axis HA1 and/or second axis HA2 substantially lean outwards away from axis LD and/or each other as first helical body 144 and second helical body 146 initially extend distal to housing distal portion 142.

In FIG. 6, first helical body 144 is configured such that ANG1 causes first axis HA1 and axis LD to define the vertex of ANG1 proximal to first supported portion 153 and/or IMD distal surface 138. For example, first axis HA1 and axis LD may define the vertex of ANG1 within a region E proximal to first supported portion 153 and/or IMD distal surface 138. In examples, first helical body 144 is configured such that the vertex of ANG1 (e.g., when proximal to first supported portion 153 and/or IMD distal surface 138) causes first base portion 152 to substantially lean away from axis LD and/or second base portion 168 as first base portion 152 extends from IMD distal surface 138 in distal direction D. In examples, region E lies proximal to (e.g. displaced in proximal direction P from) first supported portion 153 and first distal end 154.

First helical body 144 and/or housing distal portion 142 may be configured such that, as first axis HA1 becomes closer to parallel with axis LD (e.g., as ANG1 decreases as first helical body 144 travels within tissue wall 163), first helical body 144 imparts force F5 (FIG. 2) against tissues of tissue wall 163. In examples, force F5 is substantially perpendicular to first axis HA1 and/or acts in a direction substantially away from device axis LD. Force F5 may be a vector component of a resultant force exerted by first helical body 144. In examples, first distal portion 150 is configured to displace toward axis LD as first axis HA1 becomes closer to parallel with axis LD and first helical body 144 commences exerting force F5.

Stated similarly, and as depicted in FIG. 6, first helical body 144 and/or housing distal portion 142 may be configured such that, when the vertex of ANG1 (e.g., in region E) is proximal to first supported portion 153 and/or IMD distal surface 138, and in contrast to the example of FIG. 6, first point P1 is displaced from axis LD in a direction toward first side 162 (e.g., displaced in the direction RD1 (FIG. 4)). Thus, the movement of first axis HA1 in first traverse direction FT1 causes first axis HA1 to move toward axis LD (e.g., in contrast to the example of FIG. 5). The movement of first axis HA1 toward axis LD may tend to cause first helical body 144 to exert force F5 in the direction substantially away from device axis LD (e.g., due to resilient biasing of first helical body 144).

In FIG. 6, second helical body 146 is configured such that ANG2 causes second axis HA2 and axis LD to define the vertex of ANG2 proximal to first supported portion 153 and/or IMD distal surface 138. For example, second axis HA2 and axis LD may define the vertex of ANG2 within region E proximal to second supported portion 169 and/or IMD distal surface 138. In examples, second helical body 146 is configured such that the vertex of ANG2 (e.g., when proximal to second supported portion 169 and/or IMD distal surface 138) causes second base portion 168 to substantially lean away from axis LD and/or first base portion 152 as second base portion 168 extends from IMD distal surface 138 in distal direction D. In examples, region E lies proximal to (e.g., displaced in proximal direction P from) second first supported portion 169 and second distal end 156.

Second helical body 146 and/or housing distal portion 142 may be configured such that, as second axis HA2 becomes closer to parallel with axis LD (e.g., as ANG2 decreases as second helical body 146 travels within tissue wall 163), second helical body 146 imparts force F6 (FIG. 2) against tissues of tissue wall 163. In examples, force F6 is substantially perpendicular to second axis HA2 and/or acts in a direction substantially away from device axis LD. In some examples, force F6 acts in a direction substantially opposite a direction of force F5. Force F6 may be a vector component of a resultant force exerted by second helical body 146. In examples, second distal portion 166 is configured to displace toward axis LD as second axis HA2 becomes closer to parallel with axis LD and second helical body 146 commences exerting force F6.

Stated similarly, and as depicted in FIG. 6, second helical body 146 and/or housing distal portion 142 may be configured such that, when the vertex of ANG2 (e.g., in region E) is proximal to first supported portion 153 and/or IMD distal surface 138, and in contrast to the example of FIG. 6, second point P2 is displaced from axis LD in a direction toward second side 164 (e.g., displaced in the direction RD2 (FIG. 4)). Thus, the movement of second axis HA2 in second traverse direction FT2 causes second axis HA2 to move toward axis LD (e.g., in contrast to the example of FIG. 5). The movement of second axis HA2 toward axis LD may tend to cause second helical body 146 to exert force F6 in the direction substantially away from device axis LD (e.g., due to resilient biasing of second helical body 146).

Thus, first helical body 144 and/or second helical body 146 may be configured (e.g., resiliently biased) to exert one or more forces on tissues of tissue wall 163 inward toward axis LD (e.g., force F3 and/or force F4) or outward away from axis LD (e.g., force F5 and/or force F6) to, for example, assist in maintaining a fixation of IMD 102, 103, 105 to tissue wall 163 and/or maintain an orientation of axis LD relative to tissue wall 163 (e.g., tissue surface 165). First helical body 144 and/or second helical body 146 may be configured such that first axis HA1 and/or second axis HA2 move (e.g., rotate) relative to housing distal portion 142 as first helical body 144 and/or second helical body 146 implant within tissue wall 163. First helical body 144 and/or second helical body 146 may be configured such that a resilient biasing cause the exertion of the one of the more forces when first axis HA1 and/or second axis HA2 move (e.g., rotate) relative to housing distal portion 142 (e.g., as first helical body 144 and/or second helical body 146 implants within tissue wall 163.)

First base portion 152 may be coupled to and/or supported by housing distal portion 142 (e.g., IMD distal surface 138). In examples, first helical body 144 is configured to transfer a force (e.g., force F1) from first distal end 154 to first base portion 152. First base portion 152 may be configured to transfer the force (e.g., force F1) to housing distal portion 142. Second base portion 168 may be coupled to and/or supported by housing distal portion 142 (e.g., IMD distal surface 138). In examples, second helical body 146 is configured to transfer a force (e.g., force F2) from second distal end 156 to second base portion 168. Second base portion 168 may be configured to transfer a force (e.g., force F2) to housing distal portion 142.

In examples, first base portion 152 and second base portion 168 are configured to transfer countering forces to housing distal portion 142 during and/or following an implantation in tissue wall 163. For example, and referring largely to FIG. 4, housing distal portion 142 may be configured such that, if acting alone, the force (e.g., force F1) transferred by first base portion 152 might cause a movement of housing distal portion 142 causing axis LD to deviate from an orientation toward region R. In some cases, when force F1 results from the rotation of housing distal portion 142 about axis LD, the force transferred by first base portion 152, when acting alone and in concert with the rotation about axis LD, may cause a movement of housing distal portion 142 which alters a location of first distal end 154 on tissue surface 165, potentially compromising the initial orientation of housing distal portion 142 relative to tissue wall 163.

Housing distal portion 142 (e.g., of IMD 102, 103, 105) is configured to substantially counter the force transferred by first base portion 152 using second base portion 168. Housing distal portion 142 may be configured such that second base portion 168 substantially transfers its force (e.g., force F2) to housing distal portion 142 as first base portion 152 transfers its force (e.g., force F1) to housing distal portion 142. The transfer of force to housing distal portion 142 by second base portion 168 may tend to counter (e.g., to balance) the force transferred to housing distal portion 142 by first base portion 152, such that deviation of housing distal portion 142 from an initial orientation relative to tissue surface 165 may be mitigated.

Referring largely to FIG. 4, in some examples, first helical body 144 and second helical body 146 are configured such that first base portion 152 (e.g., first supported portion 153) is proximate to first side 162 of housing distal portion 142, and second base portion 168 (e.g., second supported portion 169) is proximate second side 164 of housing distal portion 142. For example, first helical body 144 and second helical body 146 may be configured such that first supported portion 153 is displaced from axis LD by a radius such as first distal radius RD1 and second supported portion 169 is displaced from axis LD by a radius such as second distal radius RD2. Thus, in some examples, first supported portion 153 and second supported portion 169 may be separated by an arc angle similar to distal arc angle A1. Hence, the arc angle separating first supported portion 153 and second supported portion 169 may be greater than about 120 degrees and less than about 240 degrees in some examples, greater than about 145 degrees and less than about 215 degrees in some examples, greater than about 170 degrees and less than about 190 degrees in some examples, and/or about 180 degrees in some examples.

A first supported portion of first helical body 144 have any angular orientation relative to first distal end 154. For example, in some examples, rather than first supported portion 153, first helical body 144 (e.g., first base portion 152) may define a first supported portion 170 defining an angular orientation described by an arc angle A2. In examples, first supported portion 170 is displaced from axis LD by a radius R3 extending from axis LD to first supported portion 170, and radius R3 and first distal radius RD1 subtend the arc angle A2 (e.g., subtend arc angle A2 in a plane substantially perpendicular to axis LD). In some examples, arc angle A2 is greater than about 45 degrees and less than about 135 degrees, although this is not required. First helical body 144 may be configured such that arc angle A2 has any value. In some examples, first supported portion 170 is configured such that arc angle A2 mitigates a bending of first helical body 144 (e.g., a bending due to movement of IMD housing 141, 127, 129).

A second supported portion of second helical body 146 have any angular orientation relative to second distal end 156. For example, in some examples, rather than second supported portion 169, second helical body 146 (e.g., second base portion 168) may define a second supported portion 172 defining an angular orientation described by an arc angle A3. In examples, second supported portion 172 is displaced from axis LD by a radius R4 extending from axis LD to second supported portion 172, and radius R4 and second distal radius RD2 subtend the arc angle A3 (e.g., subtend arc angle A3 in a plane substantially perpendicular to axis LD). In some examples, arc angle A3 is greater than about 45 degrees and less than about 135 degrees, although this is not required. Second helical body 146 may be configured such that arc angle A3 has any value. In some examples, second supported portion 172 is configured such that arc angle A3 mitigates a bending of second helical body 146 (e.g., a bending due to movement of IMD housing 141, 127, 129).

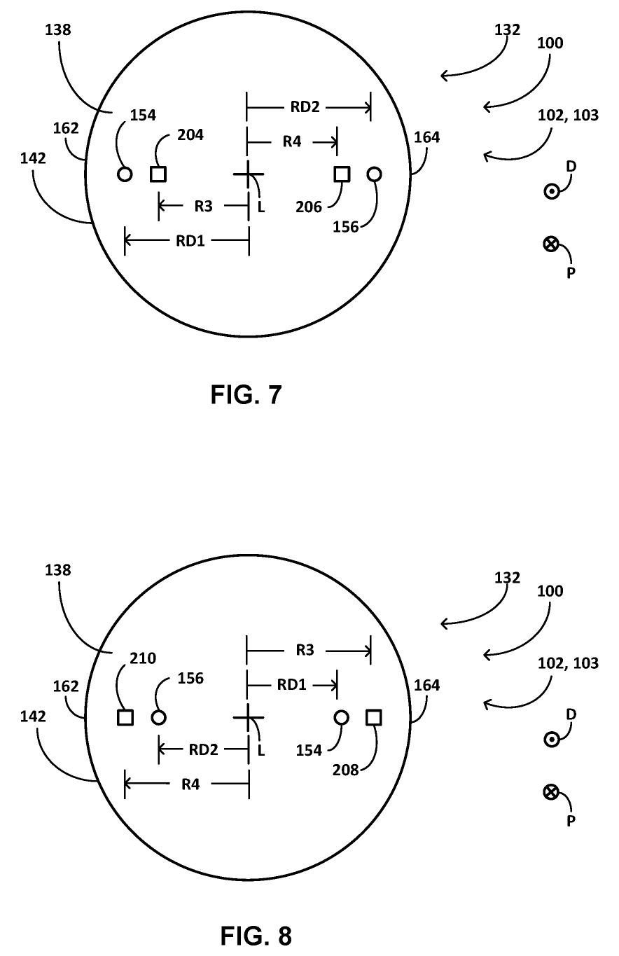

First distal end 154, first supported portion 153, second distal end 156, and/or second supported portion 169 may define any displacements relative to device axis LD and/or each other. First axis HA1 and/or second axis HA2 may define any angles relative to device axis LD. For example, FIG. 7 is schematic end view of IMD 102, 103, 105 depicting an arrangement wherein first helical body 144 extends from a first supported portion 204 to first distal end 154 and/or second helical body 146 extends from a second supported portion 206 to second distal end 154. FIG. 8 is schematic end view of IMD 102, 103, 105 depicting an arrangement wherein first helical body 144 extends from a first supported portion 208 to first distal end 154 and/or second helical body 146 extends from a second supported portion 210 to second distal end 154. First supported portion 204, 208 is an example of first supported portion 153, 170 and second supported portion 206, 210 is an example of second supported portion 169, 172.

For example, as depicted in FIG. 7, in some examples, first helical body 144 may be configured such that first distal radius RD1 from device axis LD to first distal end 154 is greater than radius R3 from device axis LD to first supported portion 204. In similar manner, in some examples, second helical body 146 may be configured such that second distal radius RD2 from device axis LD to second distal end 156 is greater than radius R4 from device axis LD to second supported portion 206. In some examples, as depicted in FIG. 8, first helical body 144 may be configured such that first distal radius RD1 from device axis LD to first distal end 154 is less than radius R3 from device axis LD to first supported portion 208. In similar manner, in some examples, second helical body 146 may be configured such that second distal radius RD2 from device axis LD to second distal end 156 is less than radius R4 from device axis LD to second supported portion 210.

As before, radius R3 may be substantially parallel to first distal radius RD1 (e.g., as depicted in FIG. 7 and FIG. 8) or may define a non-zero angle A2 (FIG. 4) with RD1, and /or radius R4 may be substantially parallel to first distal radius RD2 (e.g., as depicted in FIG. 7 and FIG. 8), or may define a non-zero angle A3 (FIG. 4) with RD1. Further, a displacement defined by first distal radius RD1, radius R3, and/or the angle A2, may be selected based on a magnitude desired for force F3 and/or force F5 (FIG. 2). A displacement defined by second distal radius RD2, radius R4, and/or the angle A3, may be selected based on a magnitude desired for force F4 and/or force F6 (FIG. 2).

As used here, a turn of a helical body (e.g., first helical body 144 or second helical body 146) may refer to a portion of the helical body which extends around a helical axis (e.g., first axis HA1 or second axis HA2) of the helical body over 360 degrees. A helix and/or helical body may refer to a body defining a corkscrew-type shape around an axis such as first axis HA1 or second axis HA2. The corkscrew-type shape may substantially spiral around the axis (e.g., a linear axis). For example, first helical body 144 may define a first corkscrew-type shape substantially spiraling around first axis HA1 and/or axis LD and extending from first base portion 152 to about first distal end 154. Second helical body 146 may define a second corkscrew-type shape substantially spiraling around second axis HA2 and/or axis LD and extending from second base portion 168 to about second distal end 156. In some examples, first helical body 144 defines a first substantially circular helix and/or second helical body 146 defines a second substantially circular helix, although this is not required. First helical body 144 and/or second helical body 146 may define a conic helix, a general helix, a cylindrical helix, a slant helix, a conic helix, a space spiral, or other like shapes which substantially spiral around an axis.

In examples, IMD distal surface 138 includes a substantially planar surface substantially perpendicular to axis LD, although this is not required. IMD distal surface 138 may include a surface defining one or more profiles of profile segments that are substantially curved, curvilinear, or linear. In some examples, IMD distal surface 138 may include a surface of revolution defined by rotating a generatrix about axis LD. In examples, IMD distal surface 138 defines a point that defines a distal-most extension of IMD housing 141, 127, 129. For example, IMD distal surface 138 may substantially define a distal end of IMD housing 141, 127, 129. IMD 102 may be configured such that first distal end 154 and second distal end 156 position distal to the point defining the distal-most extension of IMD housing 141, 127, 129. In some examples, first distal radius RD1 and/or second distal radius RD2 are substantially perpendicular to axis LD. In some examples, first distal radius RD1 and second distal radius RD2 are coplanar (e.g., such that both first distal radius RD1 and second distal radius RD2 lie within a plane substantially perpendicular to axis LD).

In some examples, first helical body 144 supports a first electrode 174 (FIGS. 2, 3, 5, 6). First helical body 144 may support first electrode 174 on first distal portion 150 or first base portion 152. In some examples, first distal portion 150 supports first electrode 174 at or in proximity to first distal end 154. In some examples, first electrode 174 is mechanically supported by first helical body 144. In some examples, first electrode 174 comprises some portion of a surface of first helical body 174. For example, first helical body 144 may include a conductive material covered at least in part by an insulative material. First electrode 174 may be a surface defined by some portion of the conductive material having the insulative material removed.

Second helical body 146 may support a second electrode 176 (FIGS. 2, 3, 5, 6). Second helical body 146 may support second electrode 176 on second distal portion 166 or second base portion 168. In some examples, second distal portion 166 supports second electrode 176 at or in proximity to second distal end 156. In some examples, second electrode 176 is mechanically supported by second helical body 146. In some examples, second electrode 176 comprises some portion of a surface of second helical body 146. For example, second helical body 146 may include a second conductive material covered at least in part by a second insulative material. Second electrode 176 may be a surface defined by some portion of the second conductive material having the second insulative material removed.

In some examples, first helical body 144 may be configured to implant first electrode 174 at a first depth within tissue wall 163 and/or second helical body 146 may be configured to implant second electrode 176 at a second depth within tissue wall 163. In some examples, the second depth is different from the first depth. In some examples, the second depth may be substantially similar to the first depth. The first depth, as used herein, may refer to a displacement of first electrode 174 within tissue wall 163 and relative to tissue surface 165. The second depth, as used herein, may refer to a displacement of second electrode 176 within tissue wall 163 and relative to tissue surface 165. First helical body 144 and/or second helical body 146 may be configured such that a depth at which first electrode 174 and/or second electrode 176 is implanted within tissue wall 163 is substantially proportional to an amount and direction of rotation of IMD housing 141127129.

In some examples, instead of or in addition to first electrode 174 and/or second electrode 176, housing distal portion 142 (e.g., IMD distal surface 138) may support a third electrode 178 (e.g., an atrial electrode) (FIGS. 2, 3, 5, 6). First electrode 174, second electrode 176, third electrode 178, and/or other electrodes of IMD 102 may be configured to establish electrical communication with tissues and/or other anatomical structures within heart 101 (e.g., tissues and/or other anatomical structures within target site 104). In some examples, IMD housing 141, 127, 129 may support a return electrode 180 (FIG. 3). Processing circuitry 134 may be configured to deliver therapy to and/or sense signals from a patient using one or more of first electrode 174, second electrode 176, third electrode 178, return electrode 180, and/or other electrodes of medica system 100.

Medical system 100 (e.g., IMD 102, 103, 105) may comprise a pacemaker such as a leadless and/or wholly intracardiac pacemaker. One or more of electrodes 139, 174, 176, 178, 180 may be electrically connected to processing circuitry 134. Processing circuitry 134 may be operably connected to operating circuitry configured to deliver therapy to a patient and/or sense physiological signals of the patient using electrodes 139, 174, 176, 178, 180. In examples, IMD 102, 103, 105 includes a retrieval structure 182 (FIG. 3) configured to releasably couple with delivery system 120 (e.g., head portion 128) to assist in, for example, delivery, deployment, and/or retrieval of IMD 102, 103, 105. In some examples, retrieval structure 182 defines a proximal end 184 of IMD housing 141, 127, 129.

Processing circuitry 134 may include fixed function circuitry and/or programmable operating circuitry. In examples, processing circuitry 134 includes circuitry configured to perform one or more functions of operating circuitry, such as therapy delivery circuitry, sensing circuitry, processing circuitry, switching circuitry, communication circuitry, and/or other circuitries. Processing circuitry 134, as well as other processors, operating circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 134 includes multiple components, such as any combination of one or more microprocessors, one or more DSPs, one or more ASICs, or one or more FPGAs, as well as other discrete or integrated logic circuitry, and/or analog circuitry.

Functions attributed to processing circuitry 134 may be embodied as software, firmware, hardware or any combination thereof. Processing circuitry 134 may include, for instance, a variety of capacitors, transformers, switches, and the like configured to perform the functions of processing circuitry 134. In examples, processing circuitry 134 may be configured to communicate with another device, such as a patient input/output device, a clinician input/output device, and/or others. Processing circuitry 134 may include any suitable hardware, firmware, software or any combination thereof for communicating with another device. In addition, processing circuitry 134 may communicate with a networked computing device and a computer network. In examples, processing circuitry 134 and/or other circuitry of medical system 100 is configured to deliver stimulation signals to and/or receive sensing signals from electrodes 139, 174, 176, 178, 180, and/or other electrodes and/or sensors within medical system 100 or external to medical system 100. Processing circuitry 134 may be configured to provide electrical signals, e.g., pacing therapy, to electrodes 139, 174, 176, 178, 180, and/or other electrodes within medical system 100. Processing circuitry 134 may be configured to receive electrical signals, e.g., sensed cardiac electrical signals, from electrodes 139, 174, 176, 178, 180, and/or other electrodes within medical system 100.

Medical system 100 (e.g., processing circuitry 134) can also include a memory configured to store program instructions, such as software, which may include one or more program modules, which are executable by processing circuitry 134. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media. In some examples, the memory includes computer-readable instructions that, when executed by processing circuitry 134 cause processing circuitry 134 to perform various functions described herein and/or other functions of processing circuitry 134.

IMD housing 141, 127, 129 may enclose processing circuitry 134 and/or other circuitry within medical system 100. IMD housing 141, 127, 129 may be configured to fluidly isolate processing circuitry 134 and/or other circuitry from an environment in contact with an exterior surface of IMD housing 141, 127, 129. In examples, IMD housing 141, 127, 129 is configured to hermetically seal an enclosure defined by IMD 102, 103, 105 and holding processing circuitry 134 and/or other circuitry. IMD housing 141, 127, 129 may be configured to define shapes that are easily accepted by the patient's body while minimizing patient discomfort. For example, portions of IMD housing 141, 127, 129 (e.g., housing distal portion 142 and/or a proximal portion of IMD housing 141, 127, 129) may define a substantially cylindrical shape with cylindrical sidewalls. In other examples, portions of IMD housing 141, 127, 129 may define substantially rectangular or other non-cylindrical shapes. IMD housing 141, 127, 129 may define shapes in which corners and edges are designed with relatively large radii, in order to present a housing having smoothly contoured exterior surfaces. In examples, attachment device 132 (e.g., first helical body 144 and/or second helical body 146) is coupled to IMD housing 141, 127, 129.

Communication links 135, 137 may be hard-line and/or wireless communications links. In some examples, communication links 135, 137 may comprise some portion of processing circuitry 134. In some examples, communication links 135, 137 comprise a wired connection, a wireless Internet connection, a direct wireless connection such as wireless LAN, Bluetooth^{TM}, Wi-Fi^{TM}, and/or an infrared connection. Communication links 135, 137 may utilize any wireless or remote communication protocol.

As used here, when a first portion of a system (e.g., medical system 100) is substantially parallel to a second portion of or an axis defined by the system, this may mean the first portion is parallel or nearly parallel to the second portion or the axis to the extent permitted by manufacturing tolerances. In some examples, when the first portion is substantially parallel to the second portion or the axis, this may mean a first vector defined by the first component of the system defines an angle of less than 10 degrees, in some examples less than 5 degrees, and in some examples less than 1 degree, with a second vector defined by the second component or the axis. When a first portion of the system is substantially perpendicular to a second portion of or an axis defined by the system, this may mean the first portion is perpendicular or nearly perpendicular to the second portion or the axis to the extent permitted by manufacturing tolerances. In some examples, when the first portion is substantially perpendicular to the second portion or the axis, this may mean that the first vector defined by the first component of the system defines an angle of at least 80 degrees, in some examples at least 85 degrees, and in some examples at least 89 degrees, with the second vector defined by the second component.

As used here, when a first portion of a system (e.g., medical system 100) supports a second portion of the system, this means that when the second portion causes a first force to be exerted on the first portion, the first portion causes a second force to be exerted on the second portion in response to the first force. The first force and/or second force may be a contact force and/or an action-at-a-distance force. For example, first force and/or second force may be mechanical force, a magnetic force, a gravitational force, or some other type of force. The first portion of the system may be a portion of the system or a portion of a component of the system. The second portion of the system may be another portion of the system or another portion of the same component or a different component. In some examples, when the first portion of the system supports the second portion of the system, this may mean the second portion is mechanically supported by and/or mechanically connected to the first portion.

As used here, when a first quantity described for a system (e.g., medical system 100) is substantially equal to a second quantity described for the system or another system, this may mean the first quantity is equal to or nearly equal to the second quantity to the extent permitted by manufacturing tolerances. In some examples, when the first quantity is substantially equal to the second quantity, this may mean a difference between the first quantity and the second quantity is less than 10% of, in some examples less than 5% of, and in some examples less than 1% of, the first quantity or the second quantity.

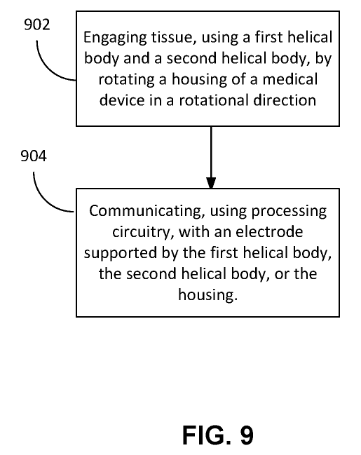

A technique for securing an implantable medical device is illustrated in FIG. 9. Although the technique is described mainly with reference to medical system 100 of FIGS. 1-6, the technique may be applied to other medical systems in other examples.

The technique includes engaging a tissue wall 163 of a patient using a first helical body 144 by rotating IMD housing 141, 127, 129 in a rotational direction (902). The rotational direction may be of a first rotational direction W1 or a second rotational direction W2. The rotation of IMD housing 141, 127, 129 relative to tissue wall 163 may cause a rotation of first helical body 144 relative to tissue wall 163. Rotation of first helical body 144 may cause a first distal end 154 to implant to a first depth within tissue wall 163. In examples, first helical body 144 surrounds an axis LD defined by IMD housing 141, 127, 129. In examples, first distal end 154 implants within tissue wall 163 over a first helical path P1 defined by first helical body 144 when first helical body 144 rotates in the rotational direction.

The technique includes engaging tissue wall 163 using a second helical body 146 using the rotation of IMD housing 141, 127, 129 in the rotational direction. The rotation of IMD housing 141, 127, 129 may cause a rotation of first helical body 144 relative to tissue wall 163. In examples, the rotation of IMD housing 141, 127, 129 causes second helical body 146 to engage tissue wall 163 substantially concurrently with first helical body 144. In examples, rotation of second helical body 146 may cause a second distal end 156 to implant to a second depth within tissue wall 163. In examples, first helical body 144 surrounds axis LD. In examples, rotating IMD housing 141, 127, 129 about axis LD causes rotation of first helical body 144 about axis LD. In examples, second distal end 156 implants within tissue wall 163 over a second helical path P2 defined by second helical body 146 when second helical body 146 rotates in the rotational direction

The technique may include causing the rotation of first helical body 144 and /or second helical body 146 by imparting a torque in the rotational direction to IMD housing 141, 127, 129. A delivery system 120 may impart the torque in the rotational direction to IMD housing 141, 127, 129. In examples, a driver 122 of delivery system 120 imparts the torque in the rotational direction to a head portion 128 of delivery system 120. Head portion 128 may transfer the torque in the rotational direction to IMD housing 141.

Engaging tissue wall 163 using first helical body 144 may include imparting a force F1 on tissue wall 163 (e.g., tissue surface 165) using first distal end 154. Engaging tissue wall 163 using second helical body 146 may include imparting a force F2 on tissue wall 163 (e.g., tissue surface 165) using second distal end 156. In examples, first helical body 144 transfers force F1 to a first base portion 152 coupled to IMD housing 141, 127, 129 (e.g., IMD distal surface 138). Second helical body 146 may transfer force F2 to a second base portion 168 coupled to IMD housing 141, 127, 129 (e.g., IMD distal surface 138). In examples, at least a component of force F2 transferred to IMD housing 141, 127, 129 counters (e.g., offsets) force F1 transferred to IMD housing 141, 127, 129.

In examples, the rotation of first helical body 144 in the rotational direction causes a first electrode 174 supported by first helical body 144 to embed within tissue wall 163. In examples, the rotation of second helical body 146 in the rotational direction causes a second electrode 176 supported by second helical body 146 to embed within tissue wall 163. In some examples, the rotation of IMD housing 141, 127, 129 causes a third electrode 178 supported by IMD housing 141, 127, 129 to contact tissue wall 163 (e.g., tissue surface 165). The technique may include communicating, using processing circuitry 134 configured to at least one provide therapy to the patient or sense a signal from the patient, with one or more of first electrode 174, second electrode 176, or third electrode 180 (904).

The technique may include reducing, as first helical body 144 implants within tissue wall 163, an angle ANG1 between a first axis HA1 of first helical body 144 and device axis LD. First helical body 144 may cause the reduction of angle ANG1 as first helical body 144 implants within tissue wall 163. In examples, the reduction of angle ANG1 causes first axis HA1 to rotate relative to IMD housing 141, 127, 129 (e.g., IMD distal surface 138). In examples, first helical body 144 moves away from axis LD when angle ANG1 reduces (e.g., when first axis HA1 rotates). In some examples, first helical body 144 moves toward axis LD when angle ANG1 reduces (e.g., when first axis HA1 rotates). First helical body 144 may impart a force F3 on tissue wall 163 (e.g., when first helical body 144 moves away from axis LD) or a force F5 on tissue wall 163 (e.g., when first helical body 144 moves toward axis LD).

The technique may include reducing, as second helical body 146 implants within tissue wall 163, an angle ANG2 between a second axis HA2 of second helical body 146 and device axis LD. Second helical body 146 may cause the reduction of angle ANG2 as second helical body 146 implants within tissue wall 163. In examples, the reduction of angle ANG2 causes second axis HA2 to rotate relative to IMD housing 141, 127, 129 (e.g., IMD distal surface 138). In examples, second helical body 146 moves away from axis LD when angle ANG2 reduces (e.g., when second axis HA2 rotates). In some examples, second helical body 146 moves toward axis LD when angle ANG2 reduces (e.g., when second axis HA2 rotates). Second helical body 146 may impart a force F4 on tissue wall 163 (e.g., when second helical body 146 moves away from axis LD) or a force F6 on tissue wall 163 (e.g., when second helical body 146 moves toward axis LD). In examples, force F4 acts in a direction opposite the direction of force F3. Force F6 may act in a direction opposite force F5.

The techniques of this disclosure may also be described in the following examples.

Example 1: A medical device configured to be positioned within an anatomical volume defined by a body of a patient, the medical device comprising: a housing configured to be positioned within an anatomical volume defined by a body of a patient, the housing defining a housing distal portion and a defining a device axis extending through the housing distal portion, wherein the housing distal portion defines a housing distal end; a first helical body configured to engage tissues, the first helical body extending in a distal direction from the housing distal portion to a first distal end distal to the housing distal end, the first helical body surrounding the device axis, and the first helical body defining a helical handedness, the handedness being one of a right-handedness or a left handedness; a second helical body configured to engage tissues as the first helical body engages the tissues, the second helical body extending in the distal direction from the housing distal portion to a second distal end distal to the housing distal end, the second helical body surrounding the device axis, and the second helical body defining the helical handedness; and processing circuitry supported by the housing, wherein the processing circuitry is configured to at least one of provide therapy to the patient or sense a signal from the patient using an electrode supported by one of the first helical body, the second helical body, or the housing.

Example 2: The medical device of claim 1, wherein the housing defines a housing proximal portion defining a housing proximal end, wherein the housing distal end and the housing proximal end are configured to position within the anatomical volume.

Example 3: The medical device of claim 1 or claim 2, wherein the first helical body defines a first helical pitch and the second helical body defines a second helical pitch, wherein the second helical pitch is substantially equal to the first helical pitch.

Example 4: The medical device of any of claims 1–3, wherein the first helical body defines a first helical diameter and the second helical body defines a second helical diameter, wherein the second helical diameter is substantially equal to the first helical diameter.

Example 5: The medical device of any of claims 1–4, wherein the first helix extends in the distal direction from a first base portion coupled to the housing distal portion and the second helix extends in the distal direction from a second base portion coupled to the housing distal portion, wherein the first distal end is displaced from the first base portion by a first helical length and the second distal end is displaced from the second base portion by a second helical length, and wherein the first helical length is substantially parallel to the second helical length.

Example 6: The medical device of any of claims 1–5, wherein the first base portion defines a first radius from the device axis and the second base portion defines a second radius from the device axis, wherein the first radius and the second radius subtend an arc angle greater than about 120 degrees and less than about 240 degrees.

Example 7: The medical device of claim 6, wherein the first radius defines a first displacement from the device axis and the second radius defines a second displacement from the device axis, wherein the first displacement is substantially equal to the second displacement.

Example 8: The medical device of claim 6 or claim 7, wherein the arc angle is substantially equal to about 180 degrees.

Example 9: The medical device of any of claims 1–8, wherein the first distal end defines a first distal radius from the device axis and the second distal end second distal radius from the device axis, wherein the first distal radius and the second distal radius subtend a distal arc angle greater than about 120 degrees and less than about 240 degrees.

Example 10: The medical device of claim 9, wherein the distal arc angle is substantially equal to about 180 degrees.

Example 11: The medical device of any of claims 1–10, wherein the first distal end is displaced from the first base portion by a first helical length and the second distal end is displaced from the second base portion by a second helical length, wherein the second helical length is substantially equal to the first helical length.

Example 12: The medical device of any of claims 1–4 or claims 6–11, wherein the first helical body defines a first helical axis, wherein the first helical axis is angularly displaced from the device axis by a first angle.

Example 13: The medical device of any of claims 1–4 or claims 6–12, wherein the second helical body defines a second helical axis, wherein the second helical axis is angularly displaced from the device axis by a second angle.

Example 14: The medical device of claim 13, wherein the first angle is substantially equal to the second angle.

Example 15: The medical device of any of claims 12–14, wherein at least one of the first angle or the second angle is less than about 30 degrees.

Example 16: The medical device of any of claims 13–15, wherein the first helical axis and the second helical axis define an angle vertex, wherein the angle vertex is distal to the housing distal portion.

Example 17: The medical device of any of claims 12–15, wherein the first helical axis and the second helical axis define an angle vertex, wherein the angle vertex is proximal to a distal surface defined by the housing distal portion.

Example 18: The medical system of any of claims 1–17, wherein the anatomical volume is defined by a volume of tissue comprising a heart of the patient.

Example 19: The medical system of any of claims 1–18, wherein the housing is configured to be transported through vasculature of the patient.

Example 20: The medical device of any of claims 1–19, wherein at least one of: the first helical electrode supports a first electrode configured to electrically connect to processing circuitry to at least one of provide therapy to the patient or sense a signal from the patient using the first electrode, the second helical electrode supports a second electrode configured to electrically connect to the processing circuitry to at least one of provide therapy to the patient or sense a signal from the patient using the second electrode, or the housing supports a third electrode configured to electrically connect to processing circuitry to at least one of provide therapy to the patient or sense a signal from the patient using the housing electrode.

Example 21: The medical device of claim 20, wherein the first helical electrode supports the first electrode at a distal end of the first helical body, and wherein the second helical electrode supports the second electrode at a distal end of the second helical body.

Example 22: The medical device of claim 20, wherein the first helical electrode supports the first electrode at a distal end of the first helical body, and wherein the second helical electrode supports the second electrode at a base portion of the second helical body.

Example 23: The medical device of claim 20, wherein the first helical electrode supports the first electrode at a base portion of the first helical body, and wherein the second helical electrode supports the second electrode at a base portion of the second helical body.

Example 24: The medical device of any of claims 20–23, wherein the housing distal end supports the third electrode.

Example 25: A medical device configured to be positioned within an anatomical volume defined by a body of a patient, the medical device comprising: a housing configured to be positioned within an anatomical volume defined by a body of a patient, the housing defining a housing distal portion and a defining a device axis extending through the housing distal portion, wherein the housing distal portion defines a housing distal end; a first helical body configured to engage tissues, the first helical body extending in a distal direction from a first base portion coupled to the housing distal portion to a first distal end distal to the housing distal end, the first helical body surrounding the device axis, and the first helical body defining a helical handedness, the handedness being one of a right-handedness or a left handedness; a second helical body configured to engage the tissues as the first helical body engages the tissues, the second helical body extending in the distal direction from a second base portion coupled to the housing distal portion to a second distal end distal to the housing distal end, the second helical body surrounding the device axis, and the second helical body defining the helical handedness, wherein the first helical body defines a first helical axis angularly displaced from the device axis by a first angle, and wherein the second helical body defines a second helical axis angularly displaced from the device axis by a second angle.

Example 26: The medical device of claim 25, wherein the first angle is substantially equal to the second angle.

Example 27: The medical device of claim 25 or claim 26, wherein at least one of the first angle or the second angle is less than about 30 degrees.

Example 28: The medical device of any of claims 25–27, wherein the first helical body defines a first helical pitch and the second helical body defines a second helical pitch, wherein the second helical pitch is substantially equal to the first helical pitch.

Example 29: The medical device of any of claims 25–28, wherein the first helical body defines a first helical diameter and the second helical body defines a second helical diameter, wherein the second helical diameter is substantially equal to the first helical diameter.

Example 30: The medical device of any of claims 25–29, wherein the first helix extends in the distal direction from a first base portion coupled to the housing distal portion and the second helix extends in the distal direction from a second base portion coupled to the housing distal portion, wherein the first distal end is displaced from the first base portion by a first helical length and the second distal end is displaced from the second base portion by a second helical length, wherein the second helical length is substantially equal to the first helical length, and wherein the first helical length is substantially parallel to the second helical length.

Example 31: The medical device of any of claims 25–29, wherein the first base portion defines a first radius from the device axis and the second base portion defines a second radius from the device axis, wherein the first radius and the second radius subtend an arc angle greater than about 120 degrees and less than about 240 degrees.

Example 32: The medical device of claim 31, wherein the first radius defines a first displacement from the device axis and the second radius defines a second displacement from the device axis, wherein the first displacement is substantially equal to the second displacement.

Example 33: The medical device of claim 31 or claim 32, wherein the arc angle is substantially equal to about 180 degrees.

Example 34: The medical device of any of claims 25–33, wherein the first distal end defines a first distal radius from the device axis and the second distal end second distal radius from the device axis, wherein the first distal radius and the second distal radius subtend an distal arc angle greater than about 120 degrees and less than about 240 degrees.

Example 35: The medical device of claim 34, wherein the first distal radius defines a first distal displacement from the device axis and the second distal radius defines a second distal displacement from the device axis, wherein the first distal displacement is substantially equal to the second distal displacement.

Example 36: The medical device of claim 34 or claim 35, wherein the distal arc angle is substantially equal to about 180 degrees.

Example 37: The medical device of any of claims 25–36, wherein the first helical axis and the second helical axis define an angle vertex, wherein the angle vertex is distal to the housing distal portion.

Example 38: The medical device of any of claims 25–37, wherein the first helical axis and the second helical axis define an angle vertex, wherein the angle vertex is proximal to a distal surface defined by the housing distal portion.

Example 39: The medical device of any of claims 25–38, wherein at least one of: the first helical electrode supports a first electrode configured to electrically connect to processing circuitry to at least one of provide therapy to the patient or sense a signal from the patient using the first electrode, the second helical electrode supports a second electrode configured to electrically connect to the processing circuitry to at least one of provide therapy to the patient or sense a signal from the patient using the second electrode, or the housing supports a third electrode configured to electrically connect to processing circuitry to at least one of provide therapy to the patient or sense a signal from the patient using the housing electrode.

Example 40: The medical device of claim 39, wherein the first helical electrode supports the first electrode at a distal end of the first helical body, and wherein the second helical electrode supports the second electrode at a distal end of the second helical body.

Example 41: The medical device of claim 39, wherein the first helical electrode supports the first electrode at a distal end of the first helical body, and wherein the second helical electrode supports the second electrode at a base portion of the second helical body.

Example 42: The medical device of claim 39, wherein the first helical electrode supports the first electrode at a base portion of the first helical body, and wherein the second helical electrode supports the second electrode at a base portion of the second helical body.

Example 43: The medical device of any of claims 39–42, wherein the housing distal end supports the third electrode.

Example 44: The medical device of any of claims 39–43, further comprising the processing circuitry.

Example 45: The medical system of any of claims 25–44, wherein the anatomical volume is defined by a volume of tissue comprising a heart of the patient.

Example 46: The medical system of any of claims 25–45, wherein at least the housing distal portion is configured to be transported through vasculature of the patient.

Example 47: A method, comprising: engaging, using a first helical body, tissues of a body of a patient, the first helical body extending in a distal direction from a housing defining a housing distal portion and a defining a device axis extending through the housing distal portion, wherein the housing distal portion defines a housing distal end, and the first helical body extending from a first base portion coupled to the housing distal portion to a first distal end distal to the housing distal end, wherein the first helical body surrounds the device axis and the first helical body defines a helical handedness, the handedness being one of a right-handedness or a left handedness; engaging, using a second helical body, the tissues of the body of the patient, the second helical body extending in the distal direction from the housing, the second helical body extending from a second base portion coupled to the housing distal portion to a second distal end distal to the housing distal end, wherein the second helical body surrounds the device axis and the second helical body defines the helical handedness; and communicating, using processing circuitry configured to at least one of provide therapy to the patient or sense a signal from the patient, with an electrode supported by one of the first helical body, the second helical body, or the housing.

Example 48: The method of claim 47, wherein at least one of: the first helical body defines a first helical pitch and the second helical body defines a second helical pitch, wherein the second helical pitch is substantially equal to the first helical pitch, the first helical body defines a first helical diameter and the second helical body defines a second helical diameter, wherein the second helical diameter is substantially equal to the first helical diameter, or the first distal end is displaced from the first base portion by a first helical length and the second distal end is displaced from the second base portion by a second helical length, wherein the second helical length is substantially equal to the first helical length, and wherein the first helical length is substantially parallel to the second helical length.

Example 49: The method of claim 47 or claim 48, further comprising: imparting, using the first distal end, a first torque in a first rotational direction around the device axis on the first base portion when the first helical body engages the tissues, and imparting, using the second distal end, a second torque in a second rotational direction around the device axis on the second base portion when the second helical body engages the tissues, wherein the second rotational direction is substantially opposite the first rotational direction.

Example 50: The method of claim 49, wherein the first torque acts at a first radial displacement from the device axis and the second torque acts at a second radial displacement from the device axis, wherein the first radial displacement is substantially equal to the first radial displacement.

Example 51: The method of any of claims 47–50, further comprising engaging the tissues using the second helical body as the first helical body engages the tissues.

Example 52: The method of any of claims 47–51, further comprising: penetrating, using the first distal end, a surface defined by the tissues; and penetrating, using the second distal end, the surface as the first distal end penetrates the surface.

Example 53: The method of any of claims 47–52, wherein engaging tissues using the first helical body comprises decreasing a first angular displacement between the device axis and a first helical axis defined by the first helical body.

Example 54: The method of any of claims 47–53, wherein engaging tissues using the second helical body comprises decreasing a second angular displacement between the device axis and a second helical axis defined by the second helical body.

Example 55: The method of any of claims 47–54, wherein the electrode is a first electrode supported by the first helical body, and further comprising communicating, using the processing circuitry, with a second electrode supported by one of the second helical body or the housing.

Example 56: The method of any of claims 47–55, wherein the anatomical volume is defined by a volume of tissue comprising a heart of the patient.

Example 57: The method of any of claims 47–56, further comprising transporting, using a delivery system, the housing through vasculature of the patient.

Example 58: The method of any of claims 47–57, wherein the first helical body defines a first helical axis angularly displaced from the device axis by a first angle, and wherein the second helical body defines a second helical axis angularly displaced from the device axis by a second angle.

Various examples of the disclosure have been described. Any combination of the described systems, operations, or functions is contemplated. These and other examples are within the scope of the following claims.

1. A medical device configured to be positioned within an anatomical volume defined by a body of a patient, the medical device comprising:
a housing configured to be positioned within an anatomical volume defined by a body of a patient, the housing defining a housing distal portion and a defining a device axis extending through the housing distal portion, wherein the housing distal portion defines a housing distal end;
a first helical body configured to engage tissues, the first helical body extending in a distal direction from the housing distal portion to a first distal end distal to the housing distal end, the first helical body surrounding the device axis, and the first helical body defining a helical handedness, the handedness being one of a right-handedness or a left handedness;
a second helical body configured to engage tissues as the first helical body engages the tissues, the second helical body extending in the distal direction from the housing distal portion to a second distal end distal to the housing distal end, the second helical body surrounding the device axis, and the second helical body defining the helical handedness; and
processing circuitry supported by the housing, wherein the processing circuitry is configured to at least one of provide therapy to the patient or sense a signal from the patient using an electrode supported by one of the first helical body, the second helical body, or the housing.

2. The medical device of claim 1, wherein the housing defines a housing proximal portion defining a housing proximal end, wherein the housing distal end and the housing proximal end are configured to position within the anatomical volume.

3. The medical device of claim 1, wherein the first helical body defines a first helical axis angularly displaced from the device axis by a first angle, and wherein
the second helical body defines a second helical axis angularly displaced from the device axis by a second angle.

4. The medical device of claim 3, wherein at least one of the first angle or the second angle is less than about 30 degrees.

5. A medical device configured to be positioned within an anatomical volume defined by a body of a patient, the medical device comprising:
a housing configured to be positioned within an anatomical volume defined by a body of a patient, the housing defining a housing distal portion and a defining a device axis extending through the housing distal portion, wherein the housing distal portion defines a housing distal end;
a first helical body configured to engage tissues, the first helical body extending in a distal direction from a first base portion coupled to the housing distal portion to a first distal end distal to the housing distal end, the first helical body surrounding the device axis, and the first helical body defining a helical handedness, the handedness being one of a right-handedness or a left handedness;
a second helical body configured to engage the tissues as the first helical body engages the tissues, the second helical body extending in the distal direction from a second base portion coupled to the housing distal portion to a second distal end distal to the housing distal end, the second helical body surrounding the device axis, and the second helical body defining the helical handedness,
wherein the first helical body defines a first helical axis angularly displaced from the device axis by a first angle, and
wherein the second helical body defines a second helical axis angularly displaced from the device axis by a second angle.

6. The medical device of claim 5, wherein the first angle is substantially equal to the second angle.

7. The medical device of claim 5, wherein at least one of the first angle or the second angle is less than about 30 degrees.

8. The medical device of claim 5, wherein the first helical body defines a first helical pitch and the second helical body defines a second helical pitch, wherein the second helical pitch is substantially equal to the first helical pitch.

9. The medical device of claim 5, wherein the first helical body defines a first helical diameter and the second helical body defines a second helical diameter, wherein the second helical diameter is substantially equal to the first helical diameter.

10. The medical device of claim 5,
wherein the first helix extends in the distal direction from a first base portion coupled to the housing distal portion and the second helix extends in the distal direction from a second base portion coupled to the housing distal portion,
wherein the first distal end is displaced from the first base portion by a first helical length and the second distal end is displaced from the second base portion by a second helical length, wherein the second helical length is substantially equal to the first helical length, and wherein the first helical length is substantially parallel to the second helical length.

11. The medical device of claim 5, wherein the first base portion defines a first radius from the device axis and the second base portion defines a second radius from the device axis, wherein the first radius and the second radius subtend an arc angle greater than about 120 degrees and less than about 240 degrees.

12. The medical device of claim 11, wherein the first radius defines a first displacement from the device axis and the second radius defines a second displacement from the device axis, wherein the first displacement is substantially equal to the second displacement.

13. The medical device of claim 11, wherein the arc angle is substantially equal to about 180 degrees.

14. The medical device of claim 5, wherein the first distal end defines a first distal radius from the device axis and the second distal end defines a second distal radius from the device axis, wherein the first distal radius and the second distal radius subtend an distal arc angle greater than about 120 degrees and less than about 240 degrees.

15. The medical device of claim 14, wherein the first distal radius defines a first distal displacement from the device axis and the second distal radius defines a second distal displacement from the device axis, wherein the first distal displacement is substantially equal to the second distal displacement.

16. The medical device of claim 5, wherein the first helical axis and the second helical axis define an angle vertex, wherein the angle vertex is distal to the housing distal portion.

17. The medical device of claim 5, wherein the first helical axis and the second helical axis define an angle vertex, wherein the angle vertex is proximal to a distal surface defined by the housing distal portion.

18. The medical device of claim 5, wherein at least one of:
the first helical electrode supports a first electrode configured to electrically connect to processing circuitry to at least one of provide therapy to the patient or sense a signal from the patient using the first electrode,
the second helical electrode supports a second electrode configured to electrically connect to the processing circuitry to at least one of provide therapy to the patient or sense a signal from the patient using the second electrode, or
the housing supports a third electrode configured to electrically connect to processing circuitry to at least one of provide therapy to the patient or sense a signal from the patient using the housing electrode.

19. A method, comprising:
engaging, using a first helical body, tissues of a body of a patient, the first helical body extending in a distal direction from a housing defining a housing distal portion and a defining a device axis extending through the housing distal portion, wherein the housing distal portion defines a housing distal end, and the first helical body extending from a first base portion coupled to the housing distal portion to a first distal end distal to the housing distal end, wherein the first helical body surrounds the device axis and the first helical body defines a helical handedness, the handedness being one of a right-handedness or a left handedness;
engaging, using a second helical body, the tissues of the body of the patient, the second helical body extending in the distal direction from the housing, the second helical body extending from a second base portion coupled to the housing distal portion to a second distal end distal to the housing distal end, wherein the second helical body surrounds the device axis and the second helical body defines the helical handedness; and
communicating, using processing circuitry configured to at least one of provide therapy to the patient or sense a signal from the patient, with an electrode supported by one of the first helical body, the second helical body, or the housing.

20. The method of claim 19, further comprising:
imparting, using the first distal end, a first torque in a first rotational direction around the device axis on the first base portion when the first helical body engages the tissues, and
imparting, using the second distal end, a second torque in a second rotational direction around the device axis on the second base portion when the second helical body engages the tissues, wherein the second rotational direction is substantially opposite the first rotational direction.
